(19) United States
(12) Patent Application Publication
(10) Pub. No.: US 2024/0280579 A1
(43) Pub. Date: Aug. 22, 2024

(54) METHODS AND SYSTEMS FOR PRESENTING DIFFERENTIAL PROTEIN EXPRESSIONS ON A GRID

(71) Applicant: Olink Proteomics AB, Uppsala (SE)

(72) Inventor: Ola CASTER, Uppsala (SE)

(21) Appl. No.: 18/582,988

(22) Filed: Feb. 21, 2024

Related U.S. Application Data
(60) Provisional application No. 63/486,193, filed on Feb. 21, 2023.

(51) Int. Cl. G01N 33/68

(52) U.S. Cl. CPC G01N 33/6803

(57) ABSTRACT

Disclosed herein is a method of building a grid for presenting associations between protein expression levels and study variables of interest. The method includes building a grid frame including different sections representing different biological pathways, assigning individual proteins to specific locations as points in the sections of the grid frame corresponding to individual proteins' function in the biological pathways, collecting detected expression levels of the individual proteins, and adjusting appearance of the points in the grid frame based on the expression levels of the individual proteins.

### CROSS-REFERENCE TO RELATED APPLICATIONS

This application claims the benefit of U.S. Provisional Application No. 63/486,193 filed Feb. 21, 2023. The above-referenced patent application is incorporated by reference in its entirety.

### TECHNICAL FIELD

The present disclosure generally relates to presenting data using grids and, more particularly, to methods and systems for building grids and presenting protein expression data on a grid using multi-level mapping.

### BACKGROUND

Understanding the dynamics of the human proteome is crucial for developing biomarkers to be used as measurable indicators for disease severity and progression, patient stratification and drug development. Multi-protein signatures of various biological states and conditions, including for complex diseases such as cancer, have potentially diverse scientific and clinical applications including but not limited to, disease classification and behavior, response to therapy, and monitoring disease activity. While technological boundaries for multi-protein detection have been pushed in recent years, an effective means of presenting differences in protein expression between cohorts in complex conditions and diseases, that facilitates disease understanding, examination, diagnosis and/or treatment is generally missing, especially an effective means for presenting variations in protein expression in an intuitive way that allows directly observing, evaluating, and/or comparing expression levels of multiple proteins in an individual or a cohort of individuals and connecting these variations to actual biological pathways.

### SUMMARY

Methods and systems disclosed herein address the above problems by building a grid for presenting differences in protein expression for various health states, such as diseases, including treated and untreated. According to one aspect, the present disclosure relates to a method of building a grid for presenting differential protein expression, the method comprising: building a grid frame including different sections representing different biological pathways; assigning individual proteins to specific locations as points in the sections of the grid frame corresponding to individual proteins' function in the biological pathways; collecting detected expression levels of the individual proteins from at least two subject cohorts; and adjusting appearance of the points in the grid frame based on the difference in expression levels of the individual proteins between the at least two subject cohorts.

According to another aspect, the present disclosure relates to a method of a grid-based examination of differential protein expression, the method comprising: collecting protein expression levels of individual proteins for at least a first and a second subject cohorts; building a grid frame including different sections representing different biological pathways; assigning individual proteins into specific locations as points in the sections of the grid frame corresponding to the individual proteins' function in the biological pathways; adjusting appearance of the points in the grid frame based on the difference in expression levels of the individual proteins between the first and second subject cohorts; and examining differential protein expression of the first and second subject cohorts based on the appearance of the points.

According to another aspect, the present disclosure relates to a grid for presenting differential protein expression, the grid comprising: a plurality of sections corresponding to different biological pathways; and one or more points in each of the plurality of sections, each of the one or more points corresponding to a specific protein, each of the one or more points having an appearance corresponding to a protein expression level of the corresponding protein in a first subject cohort relative to a protein expression level of the same protein in a second subject cohort.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Furthermore, the claimed subject matter is not limited to embodiments that solve any or all disadvantages noted in any part of this disclosure.

### BRIEF DESCRIPTION OF THE DRAWINGS

These and other features, aspects, and advantages of the present disclosure will become better understood with regard to the following description and accompanying drawings. It is noted that wherever practicable similar or like reference numbers may be used in the figures and may indicate similar or like functionality. For example, a letter after a reference numeral, such as “third party entity 110A,” indicates that the text refers specifically to the element having that particular reference numeral. A reference numeral in the text without a following letter, such as “third party entity 110,” refers to any or all of the elements in the figures bearing that reference numeral (e.g., “third party entity 110” in the text refers to reference numerals “third party entity 110A” and/or “third party entity 110B” in the figures).

FIG. 1A depicts an overall system environment including a disease examination system, in accordance with an embodiment.

FIG. 1B depicts a block diagram of the disease examination system, in accordance with an embodiment.

FIG. 2 depicts example modules included in a grid generation module, in accordance with an embodiment.

FIGS. 3A-3G depicts example grids showing different features, in accordance with an embodiment.

FIGS. 4A-4N depicts example grids for different types of cancer, in accordance with an embodiment.

FIG. 5 illustrates an example method for building a grid for presenting differential protein expression using multi-level grid-mapping, in accordance with an embodiment.

FIG. 6 illustrates an example computer device for building a grid for presenting differential protein expression using multi-level grid-mapping, in accordance with an embodiment.

FIGS. 7A and 7B illustrate details of a cancer patient cohort.

FIG. 7C illustrate a plot of a Principal Component Analysis of protein expression measurements.

FIG. 7D is an illustration of up- and down-regulated proteins in certain cancer forms.

FIG. 7E is an illustration of the most significantly differentially expressed proteins in certain cancer forms.

FIGS. 7F-7H illustrate heatmaps indicating Normalized Enrichment Score and p-values for proteins relevant to certain cancer forms and related to various hierarchical levels of certain biological pathways.

FIG. 7I illustrates an example volcano plot for ANOVA-based differential expression results for acute myeloid leukemia.

FIG. 7J depicts an example grid in accordance with an embodiment.

### DETAILED DESCRIPTION

In the following detailed description of embodiments, reference is made to the accompanying drawings which form a part hereof, and which are shown by way of illustrations. It is to be understood that features of various described embodiments may be combined, other embodiments may be utilized, and structural changes may be made without departing from the spirit and scope of the present disclosure. It is also to be understood that features of the various embodiments and examples herein can be combined, exchanged, or removed without departing from the spirit and scope of the present disclosure. In addition, reference numerals and descriptions of redundant elements between figures may be omitted for clarity.

According to an embodiment, the methods and functions described herein, such as building a grid for presenting differential protein expression, may be implemented as one or more software programs running on a computer processor (e.g., a control unit or controller). According to an embodiment, the methods and functions described herein may be implemented as one or more software programs or firmware programs running on a standalone computing device or embedded apparatus, such as a tablet computer, smartphone, personal computer, server, or any other computing device, or on an appliance or apparatus with a controlling program. Dedicated hardware embodiments including, but not limited to, application-specific integrated circuits, programmable logic arrays, and other hardware devices can likewise be constructed to implement the methods and functions described herein. Further, the methods described herein may be implemented as a device, such as a non-transitory computer-readable storage medium or memory device, including instructions that when executed cause a processor to perform the methods and functions described herein.

According to an embodiment, the methods and systems disclosed herein may relate to a system for examination of differential protein expression between at least two cohort of subjects, where the system utilizes a grid for presenting relative protein expression levels in known or unknown diseases, where the grid includes different sections for different biological pathways. A biological pathway is a series of actions among molecules in a cell that leads to a certain product or a change in the cell. It can trigger the assembly of new molecules, such as a fat or protein, turn genes on and off, or spur a cell to move. Proteins are involved in biological pathways in numerous ways, such as enzymes, signal molecules, receptors etc.

According to an embodiment, the methods and systems disclosed herein may relate to a disease examination system that utilizes a grid for presenting abnormal protein expression in certain diseases. According to an embodiment, a grid for abnormal protein expression presentation may include different sections for different biological pathways.

In some embodiments, each section of the grid may be comprised of a number of polygons, which can be one to ten or even a larger number of polygons depending on the number of proteins included in a pathway. While the presently preferred shape of grid sections is hexagons, other geometric shapes are possible, such as polygons having 3, 4, 5, 7, 8, 9, 10, 11, 12 or more sides, with 3, 4, and 6 sides being preferred. In some embodiments, the grid is comprised of a mix of differently shaped sections, such as a mix of different polygons, such as octagons and squares. Under certain circumstances, a section corresponding to a biological pathway may be further divided into different subsections, so as to organize proteins involved in a pathway into different sub-groups based on the functions of these proteins or processes involved by these proteins in the pathway (e.g., in a same sub-pathway). Within each subsection or within each section if there is no subsection, the included proteins can be assigned to specific locations (e.g., randomly assigned locations) and presented as points in the assigned locations within the section/subsection. In some embodiments, to allow to present differential protein expression in a grid, the protein expression levels for individual proteins can be detected, and the size and color of a point representing a protein can be adjusted to reflect the protein expression level for that specific protein. For example, when a protein has a larger degree of upregulation or downregulation when compared to healthy individuals, the protein has a larger size of the corresponding point in the grid. In addition, different colors and/or color intensities can be utilized to differentiate upregulated protein expression levels and downregulated protein expression levels in the grid. In this way, abnormal protein expression levels for a state, condition, or disease (which can be also referred to as “multi-protein signatures” for the state, condition, or disease) can be presented in a grid.

A cohort of subjects include at least one subject, but may include any number of subjects. The subjects may be human individuals, but the present disclosures is also applicable to other species, such as mice, rats, primates etc. All members of a cohort should share one or more common traits, such as having been diagnosed with a certain disease, not being diagnosed with a certain disease, being at a certain stage of a disease, being treated or not treated with a certain drug, being generally regarded as healthy, etc. In certain embodiments, two cohorts may consist of samples from the same individuals taken at separate points in time.

It is to be understood that the features, benefits, and advantages described herein are not all-inclusive, and many additional features and advantages will be apparent to one of ordinary skill in the art in view of the figures and the following descriptions.

### System Overview

According to an embodiment of the disclosure, a grid may be built for a first cohort of subjects based on the detected protein expression relative to a second cohort, in a bodily fluid, cell or tissue lysate, dried blood spot, biopsy, tissue, organ, or another part of the patient(s). The bodily fluid may be, but is not restricted to, plasma, serum, blood, cerebrospinal fluid, saliva, urine, interstitial fluid, peritoneal fluid, breast milk, bronchoalveolar lavage fluid, and synovial fluid. The generated grid may allow a presentation of normal or abnormal protein expression levels in the members of the first cohort as compared to the second cohort if there are any. Such abnormal protein expression information can be utilized in the examination or investigation of what biological pathways are affected by abnormal protein expression in a health state, condition or disease, which facilitates understanding of disease mechanisms and/or further selection of drug targets for treatment. In an embodiment where the first cohort consist of a single member, the abnormal protein expression information may also facilitate the examination or aid diagnosis of certain diseases including certain stages of the diseases. In one example, a grid built for a patient may be used to compare to other existing grids (e.g., a library of grids generated for known disease types/stages), so as to diagnose whether the patient has a disease and/or which stage if s/he does.

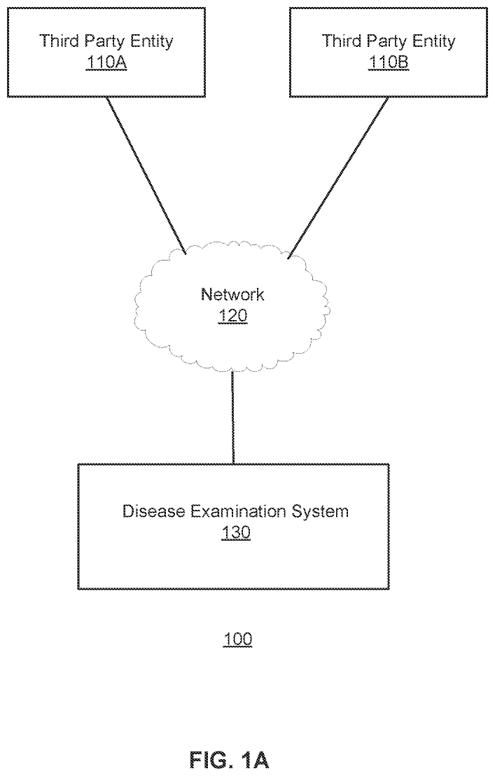

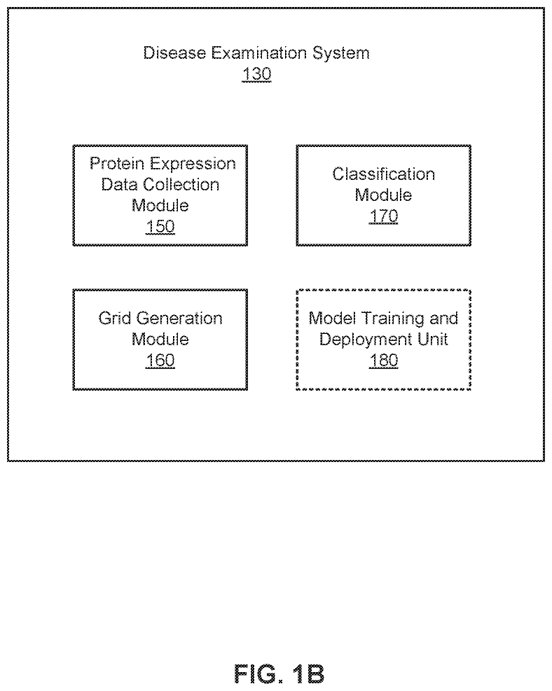

FIG. 1A depicts an overall system architecture 100 including a disease examination system 130, in accordance with an embodiment. FIG. 1A further introduces one or more third party entities 110A and 110B in communication with one another and/or the disease examination system 130 through a network 120. FIG. 1A depicts one embodiment of the overall system architecture 100. In other embodiments, additional or fewer third party entities 110 in communication with the disease examination system 130 can be included.

Generally, the disease examination system 130 performs methods disclosed herein, such as methods for generating grids for presenting differential protein expression for certain diseases and using grids for disease characterization, diagnosis, or other different purposes. For example, the grids can be built for patient cohorts based on the protein expression levels for individual proteins detected for patients. If protein expression data for a cohort of large number (e.g., hundreds or thousands) of patients of a known disease type and/or disease stage are used to generate a grid compared to healthy controls, such grid may represent differential protein expression levels of individual proteins for such disease type/stage.

In various embodiments, the methods described herein as being performed by the disease examination system 130 can be dispersed between the disease examination system 130 and third party entities 110. For example, a third party entity 110A or 110B can generate protein expression data and/or provide biological pathway models included in the system architecture 100. The data and models can be then deployed to the disease examination system 130 for examination purposes or other analysis purposes.

Referring to the third party entities 110, in various embodiments, a third party entity 110 represents a partner entity of the disease examination system 130. For example, the third party entity 110 can operate either upstream or downstream of the disease examination system 130. In various embodiments, a first third party entity 110A can operate upstream of the disease examination system 130 and a second third party entity 110B can operate downstream of the disease examination system 130.

As one example, a third party entity 110 can be an entity (such as a multiplex proteomics platform) that detects protein expression levels for individuals or an entity that provides normalized protein expression levels for individuals.

As another example, the third party entity 110 operates downstream of the disease examination system 130. In this scenario, the third party entity 110 may transmit a grid generated by the disease examination system 130 to a relevant party. In some embodiments, the third-party entity 110 may perform additional analysis for a grid, which may include, but is not limited to, identifying certain biology pathways that are related to an identified disease type/stage, identifying certain unusual protein expression patterns that are specific to a cohort, etc.

Referring to the network 120 shown in FIG. 1A, this disclosure contemplates any suitable network 120 that enables connection between the disease examination system 130 and third party entities 110. The network 120 may comprise any combination of local area and/or wide area networks, using both wired and/or wireless communication systems. In one embodiment, network 120 uses standard communications technologies and/or protocols. For example, network 120 includes communication links using technologies such as Ethernet, 802.11, worldwide interoperability for microwave access (WiMAX), 3G, 4G, code division multiple access (CDMA), digital subscriber line (DSL), etc. Examples of networking protocols used for communicating via network 120 include multiprotocol label switching (MPLS), transmission control protocol/Internet protocol (TCP/IP), hypertext transport protocol (HTTP), simple mail transfer protocol (SMTP), and file transfer protocol (FTP). Data exchanged over network 120 may be represented using any suitable format, such as hypertext markup language (HTML) or extensible markup language (XML). In some embodiments, all or some of the communication links of network 120 may be encrypted using any suitable technique or techniques.

FIG. 1B depicts a block diagram of the disease examination system 130, in accordance with an embodiment. FIG. 1B introduces individual modules of the disease examination system 130 which includes, in various embodiments, a protein expression data collection module 150, a grid generation module 160, and a classification module 170. In some embodiments, the disease examination system 130 optionally includes a model training and deployment unit 180.

Generally, the protein expression data collection module 150 is in charge of data collection of protein expression data for all or some of the members of the at least two cohorts of subjects (e.g., patients). The data collection module 150 may collect protein expression data for subjects (e.g., patients or healthy individuals) from different entities, e.g., different organizations or institutes. In some embodiments, these different organizations or institutes may perform certain analyses on protein expression levels. According to one example, an organization or institute may collect plasma samples from cancer patients around the time of diagnosis or at certain stages of the disease, and from healthy controls. The collected samples may be then subject to analysis by using a multiplex proteomics platform (e.g., Olink® Explore) for detecting individual protein expression levels. In some embodiments, the protein expression data collection module 150 may optionally include a data analysis module configured to perform certain data analysis if the data collected by the collection module 150 is raw data, as further described in detail below.

In some embodiments, the measured protein expression levels may be converted to relative values (e.g., normalized protein expression levels expressed as NPX values) or presented as ANOVA (analysis of variance) estimates, or absolute values thereof, as will be described later. NPX values can be calculated from protein expression data obtained using Olink® Target, Olink® Focus, Olink® Flex and Olink® Explore and accompanying dedicated software (Olink Proteomics AB, Uppsala, Sweden). For example, a measured difference of 1 NPX between two measurements (e.g., between patients and healthy individuals, or between patients of cancer A vs patients of cancer B-cancer N) for a same protein assay may represent approximately a doubling of the protein concentration.

In some embodiments, the data analysis module may perform further statistical analysis for the detected protein expression levels. For example, to identify proteins that are different in their NPX levels between different types of cancers, a standard differential expression analysis may be performed by fitting one ANOVA model per protein. Under certain circumstances, certain data points can be removed due to a quality control warning and replaced with other reference values (e.g., media values from the measurements) during the analysis. In addition, certain analyses may be adjusted for relevant covariates, such as age and, when applicable, sex.

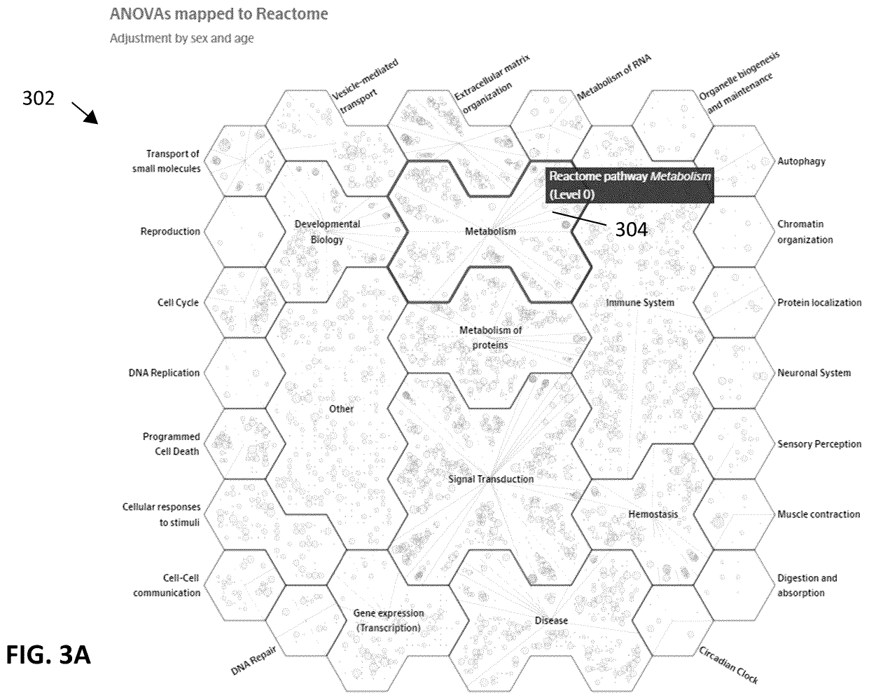

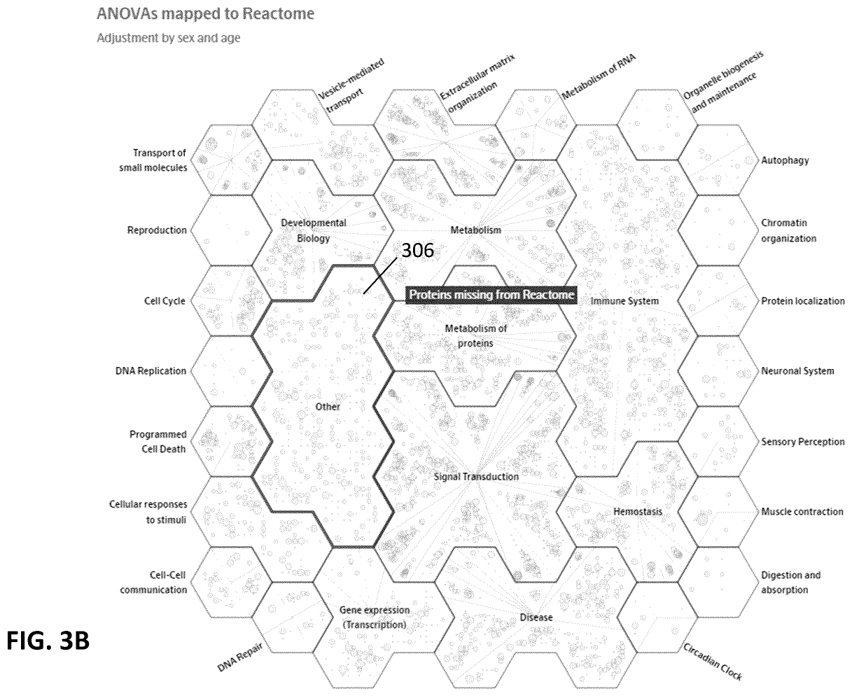

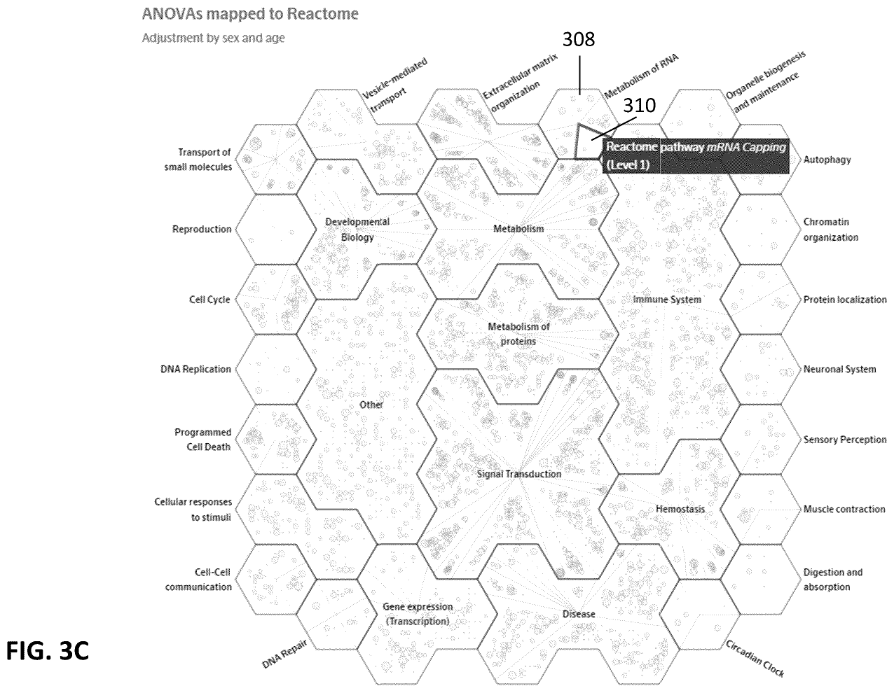

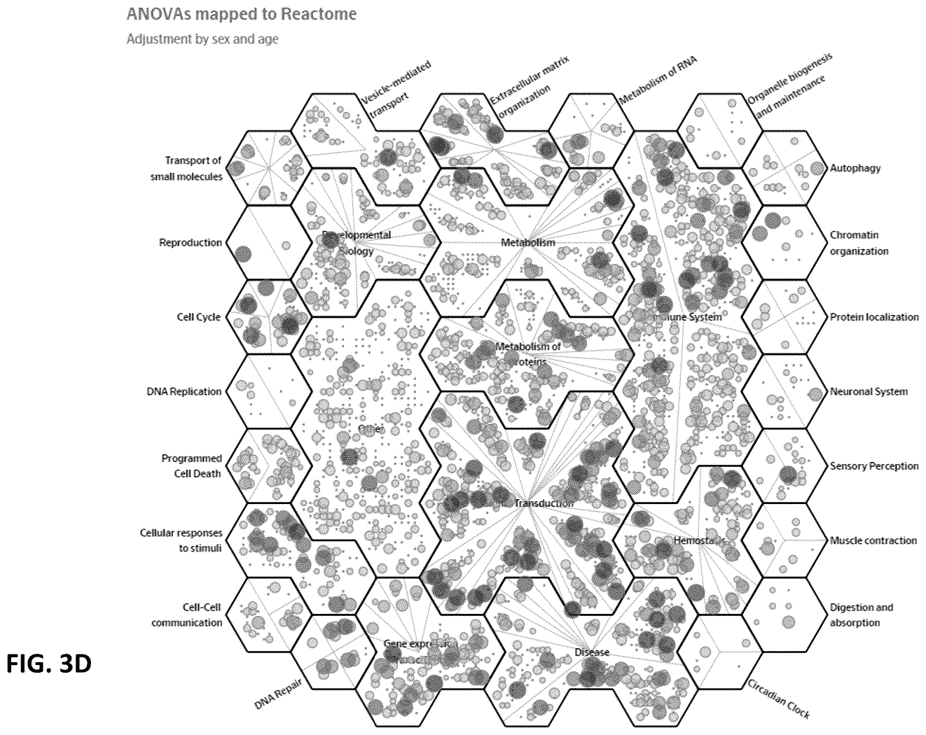

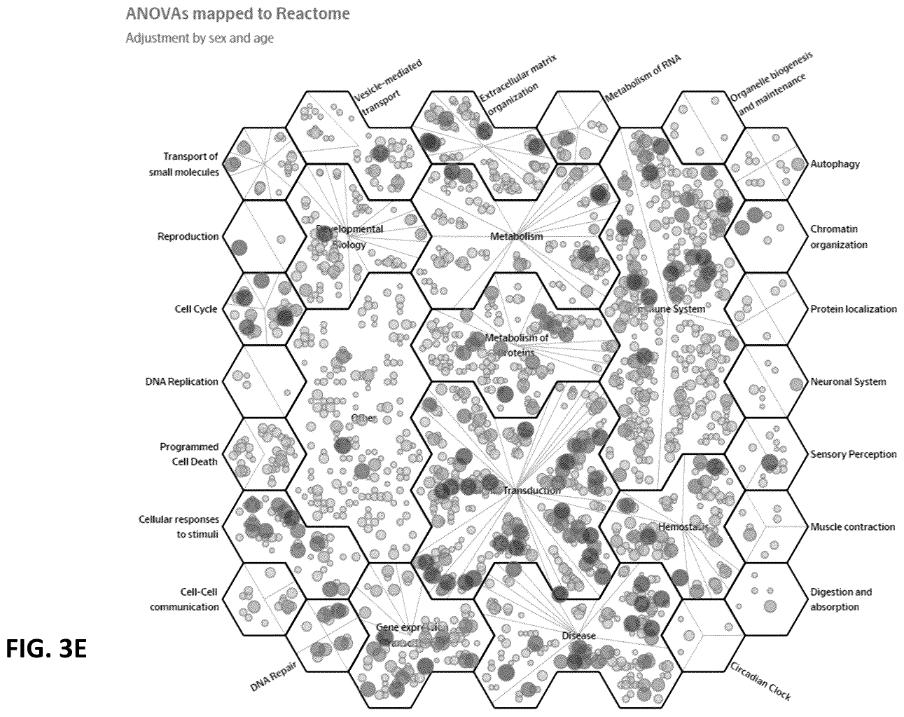

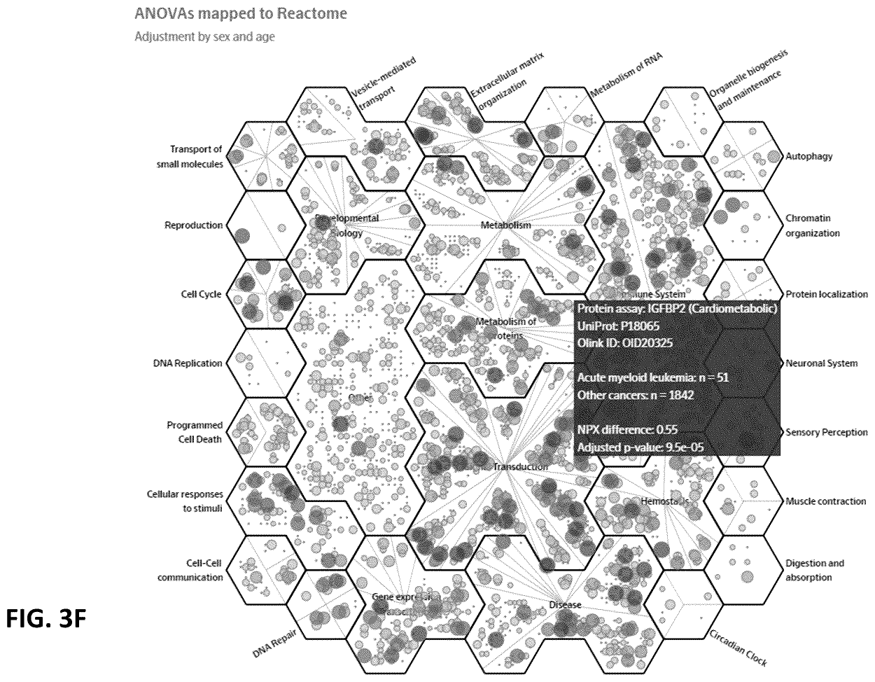

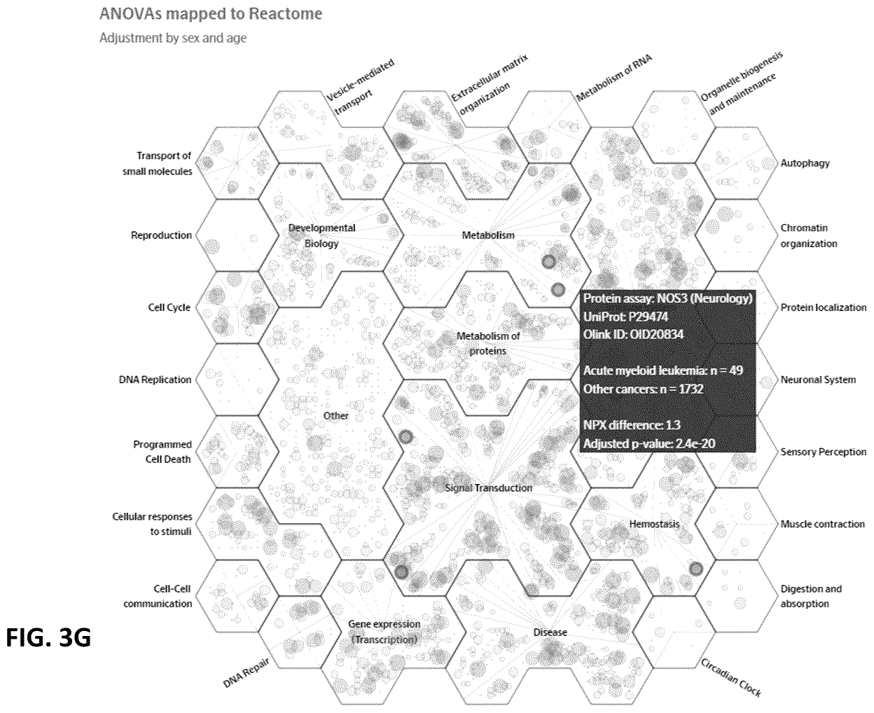

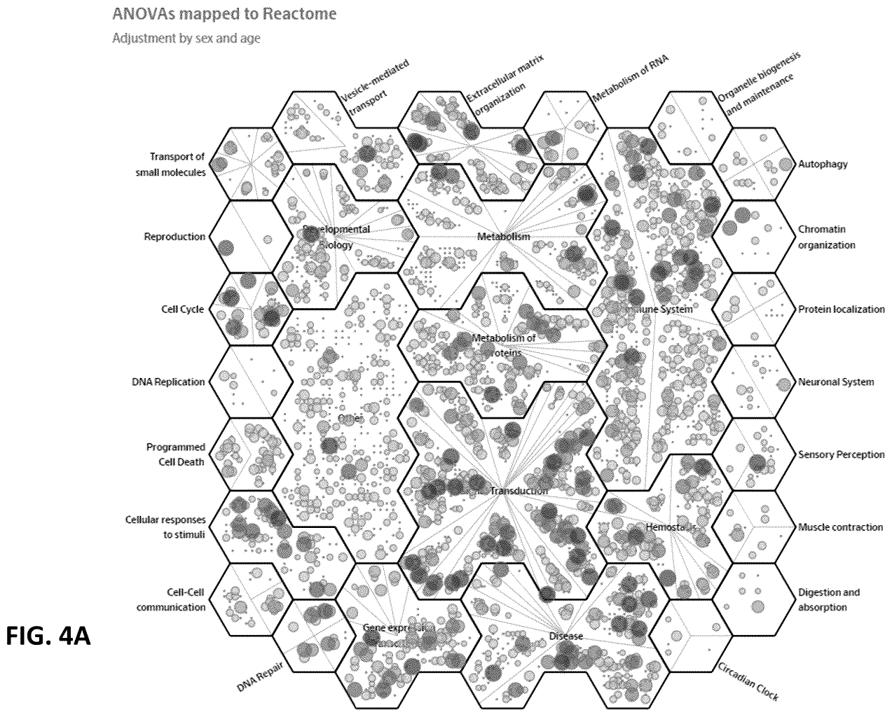

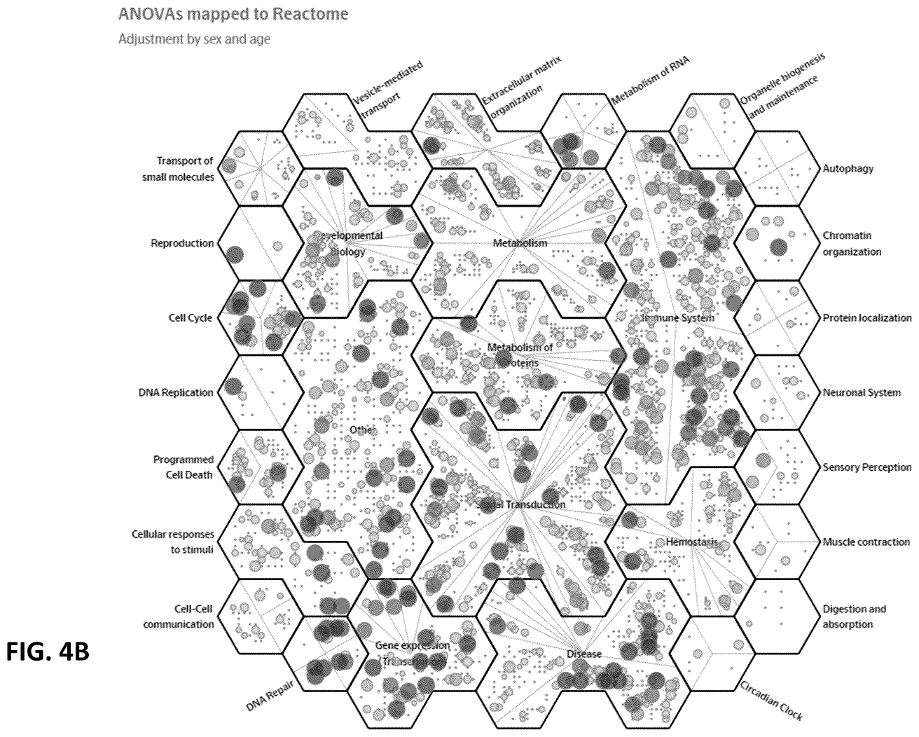

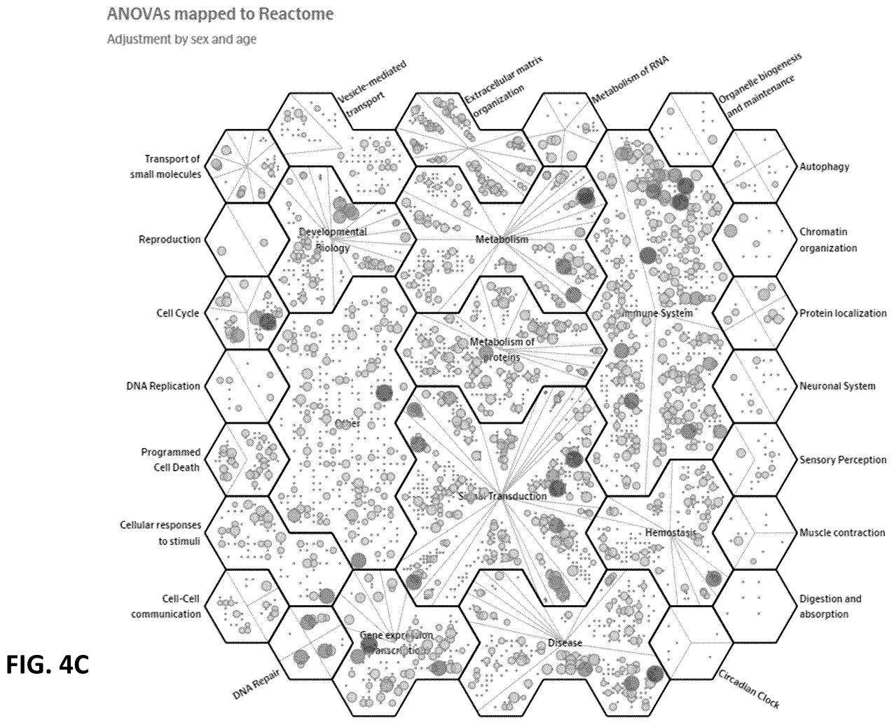

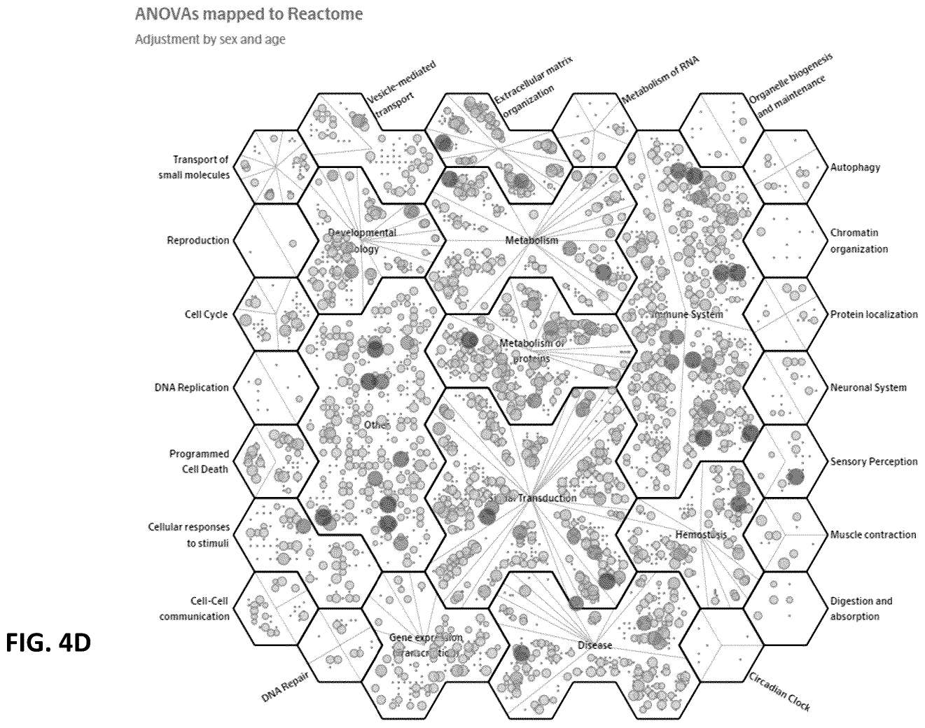

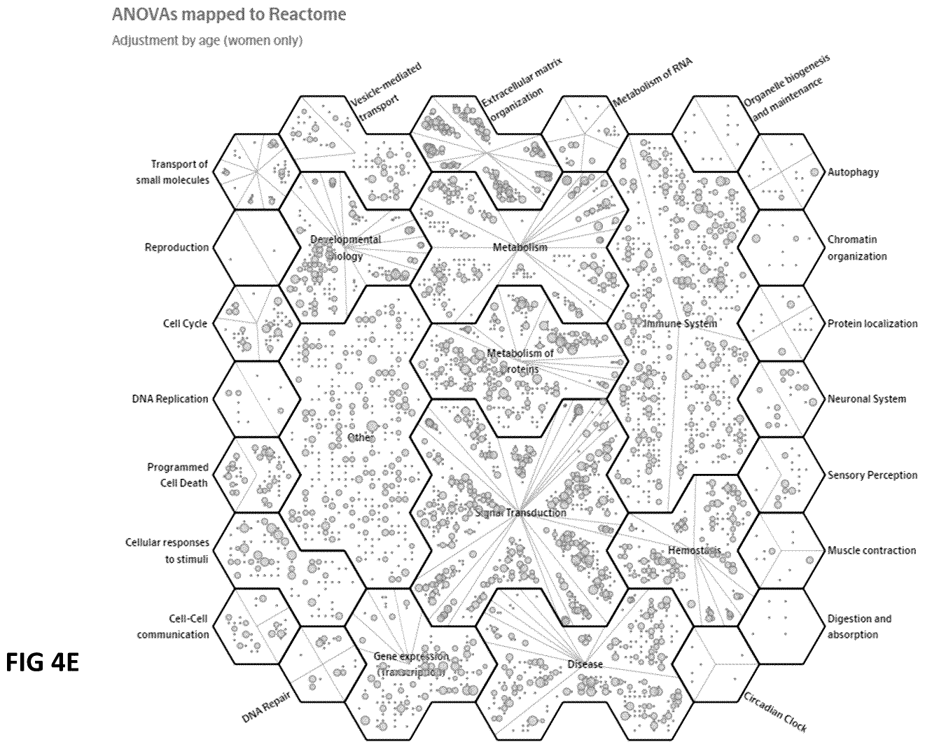

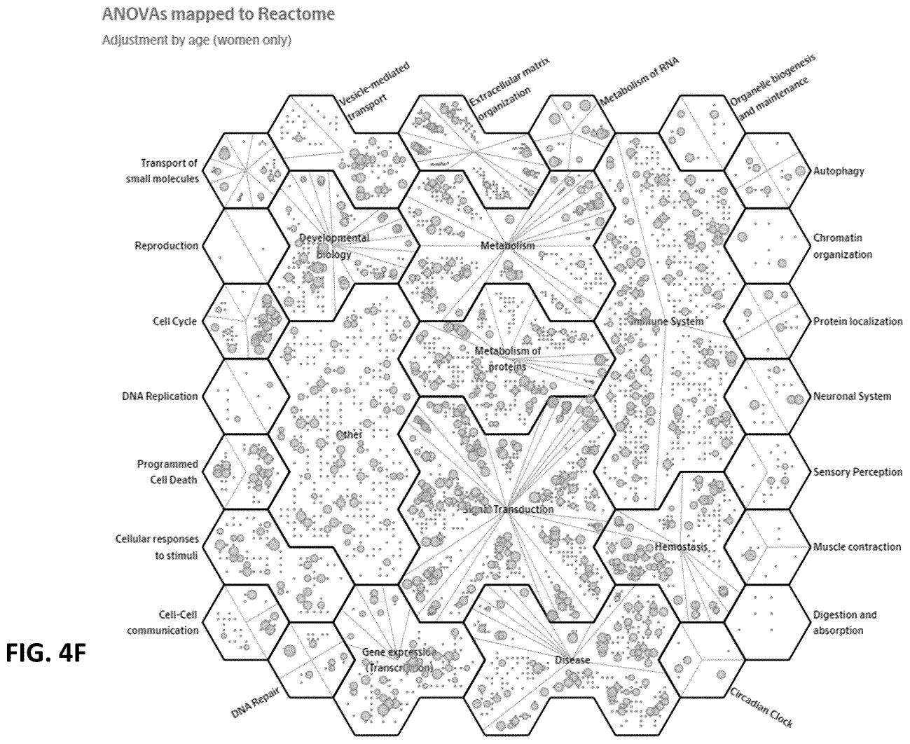

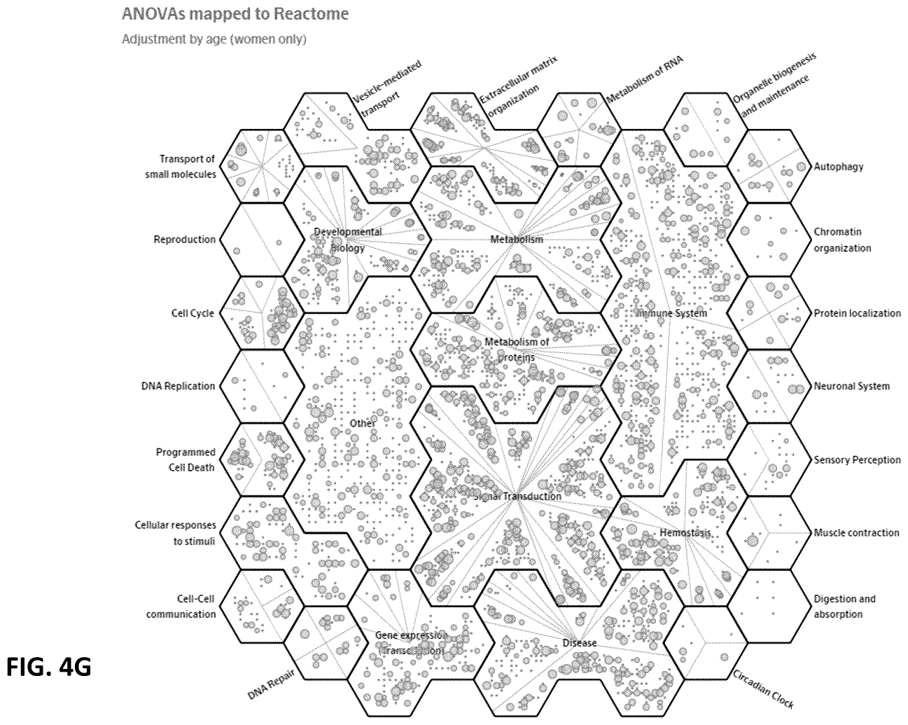

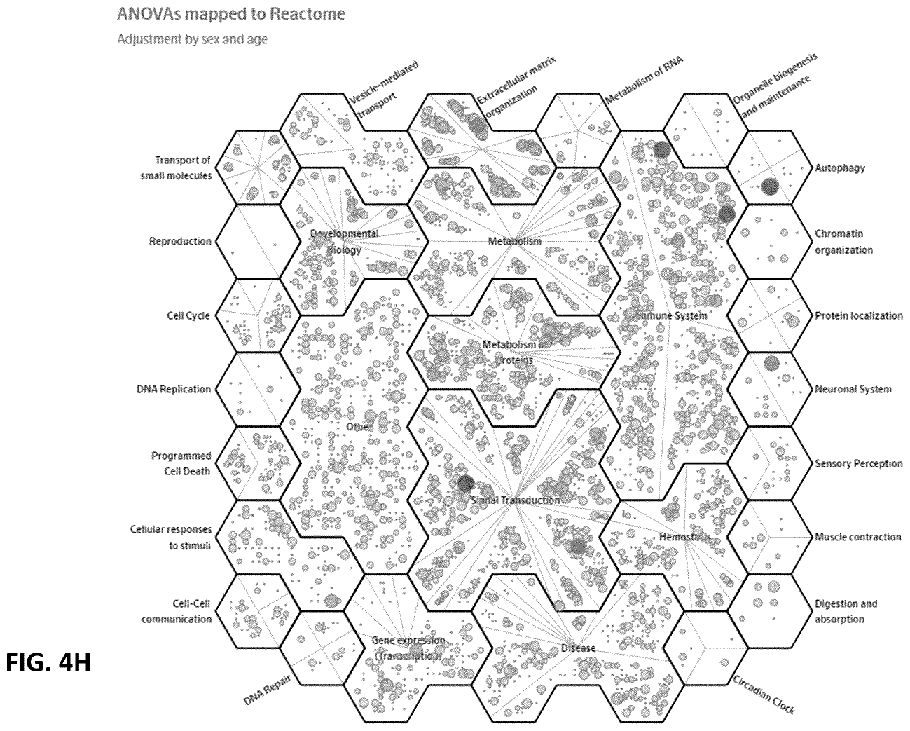

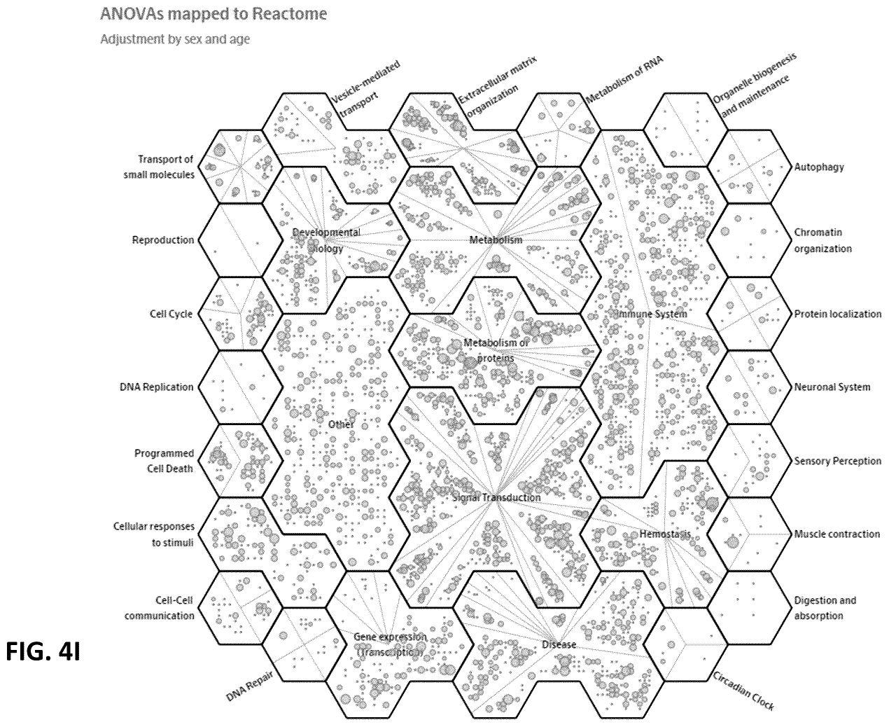

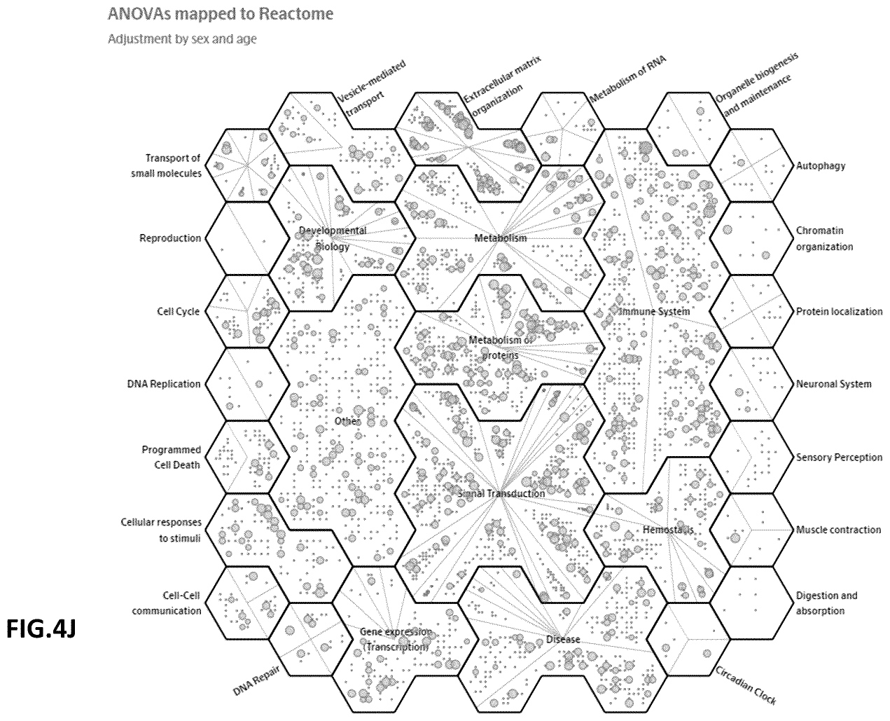

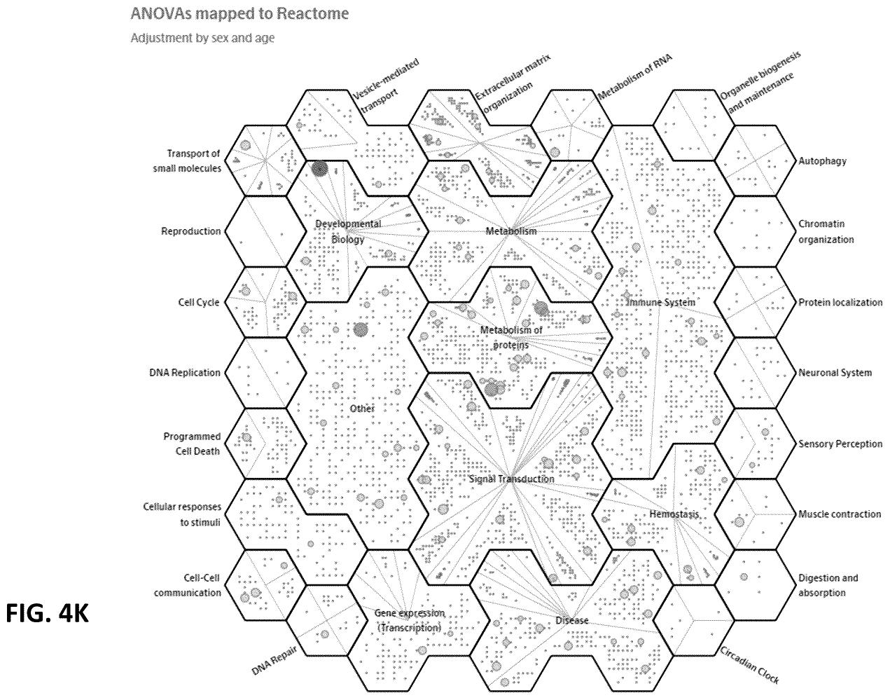

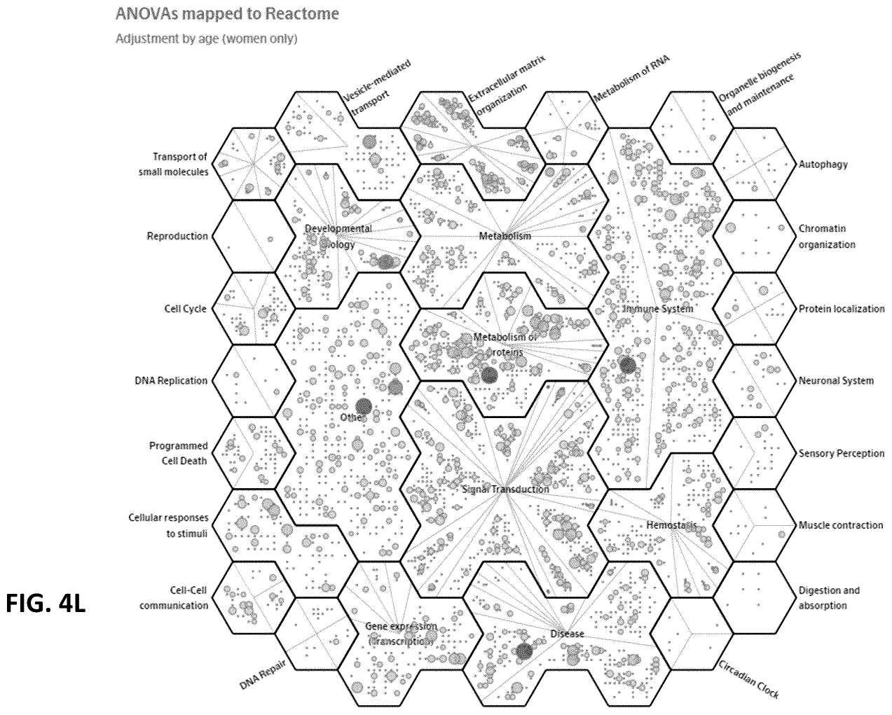

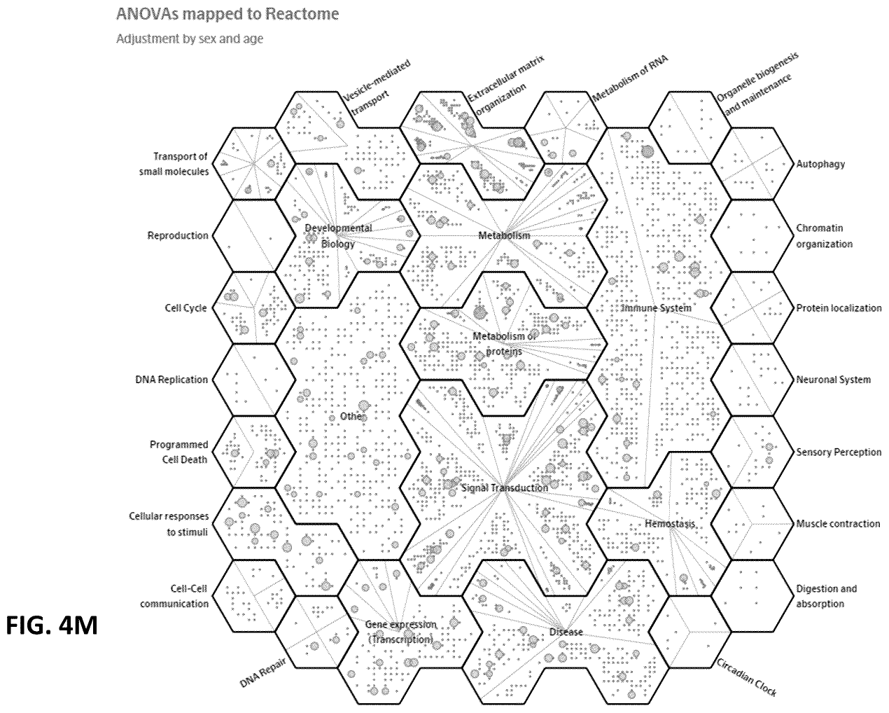

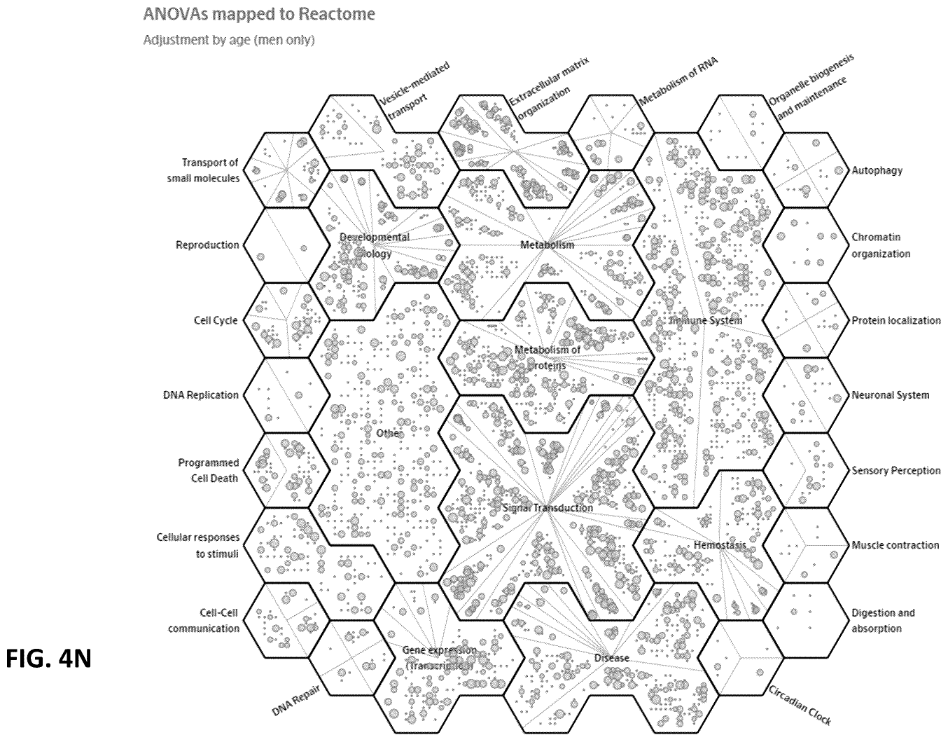

The grid generation module 160 is configured to build or generate a grid based on differential protein expression detected for a type of disease or for two or more cohorts. As described elsewhere herein, a grid may be a graph that includes a certain number of polygons (e.g., hexagons as shown in FIGS. 3A-4N) organized in a map format. These polygons can be organized into different sections and/or subsections, where a section corresponds to a biological pathway and a subsection may correspond to a subset of proteins that relate to similar functions in the biological pathway or a same or relevant process(es) in the pathway (e.g., in a same sub-pathway).

In some embodiments, to build a graph with map features, the grid generation module 160 further assigns proteins included in a pathway to specific locations as individual points in the corresponding sections/subsections. In some embodiments, the grid generation module 160 further adjusts appearance including but not limited to the size and color of the individual points according to the differential expression of individual proteins between cohorts. In one example, the grid generation module 160 may adjust the size of a point for a protein based on the NPX value determined for the protein in one cohort as compared to that determined for the same protein in another cohort. In another example, the grid generation module 160 may adjust the color of a point based on whether the protein expression of the corresponding protein is upregulated or downregulated. An upregulated protein expression may have a different color or color intensity from a downregulated protein expression. After generation, the grid may then reflect the relative protein expression levels for individual proteins. The larger size of a point, the more obvious change in protein expression for a specific protein.

In some embodiments, the grid generation module 160 may be configured to generate a grid library that includes a plurality of grids with known disease types and/or stages. These grids in the library may be generated based on a large number of samples with known disease types/stages. In some embodiments, the grids generated in the library may be dynamically updated. For example, when more or more additional samples are collected, the newly collected data may be used to adjust a grid in the library if necessary. For example, the size of a point in a grid may be adjusted based on the detected protein expressions for certain new subjects.

The classification module 170 is configured to classify proteins for which protein expression data is available as being part of one or more biological pathways. In one example, the classification module 170 may be configured to access information on involvement of proteins in biological pathways from an external database and classifying a protein for which protein expression data is available as involved in a certain biological pathway according to the accessed information.

In some embodiments, a machine learning-based model may be developed and included in the classification module 170. The machine learning-based model may be trained with relevant biological and biochemical data, such as data related to amino acid sequence, protein function, and molecular interactions in organisms. The training may include a testing stage and an evaluation stage to make sure proper biological pathways be identified after the training process. Once trained, the machine learning-based model can be then utilized for the classification of proteins to biological pathways, e.g., by feeding the amino acid sequence of the protein into the trained machine learning-based model in the classification module 170 for classification to a biological pathway.

In some embodiments, the disease examination system 130 may optionally include a model training and deployment module 180. The model training and deployment module 180 may be configured to train the above-described machine learning-based model. Once the model is trained, the model training and deployment module 180 may then deploy the model for protein classification. In some embodiments, the model training and deployment module 180 may be also configured to train and deploy certain other models included in the disease examination system 130. For example, the model training and deployment module 180 may also train a model for data analysis (e.g., lasso regression), according to an embodiment.

In some embodiments, the disease examination system 130 may include fewer or additional components than those described above. In one example, the disease examination system 130 may not include a classification module 170 and/or a model training and deployment module 180, and thus may be simply referred to as a grid generation system 130.

### Grid Generation Implementation

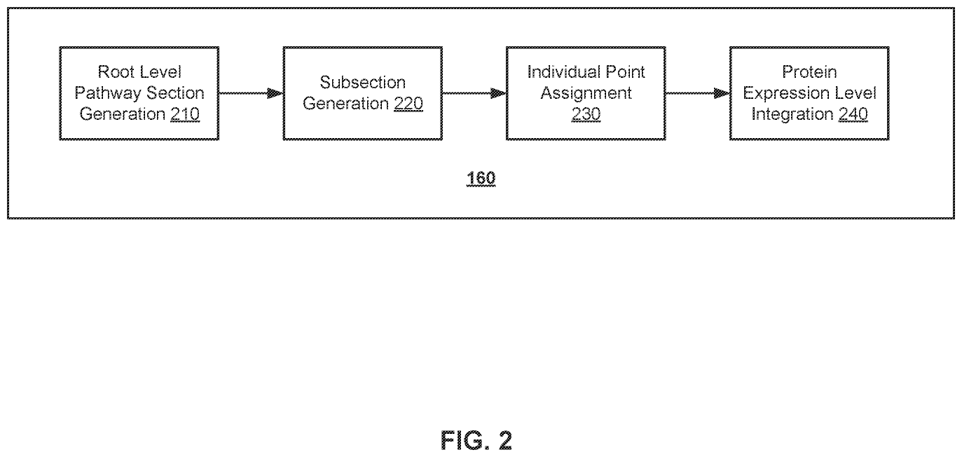

Referring now to FIG. 2, an example process for building a grid for presenting normal or abnormal protein expression is further described. The process may be implemented by the above-described grid generation module 160. According to an embodiment, the process may start with a root level pathway section generation 210, to generate a root level grid frame that presents different biological pathways in polygon format. Here, the root level may be also referred to as “level 0.” In the root level biological pathway frame, each pathway may be represented by a section including a set of polygons (e.g., hexagons).

FIG. 3A illustrates an example root level biological pathway frame 302 that presents biological pathways in a hexagon format, according to an embodiment. In the illustrated embodiment, the pathways utilized as examples of biological pathways in the grid format are based on biological pathway data from the Reactome database (reactome.org) (the pathways together or individually may be also referred to as Reactome pathway). As can be seen in FIG. 3A, pathways presented in the root level grid frame include pathways for the Transport of Small Molecules, Reproduction, Cell Cycle, DNA Replication, Programmed Cell Death, Cellular Response to Stimuli, Cell-Cell Communication, DNA repair, Developmental Biology, Gene Expression (Transcription), Vehicle-Mediated Transport, Extracellular Matrix Organization, Metabolism, Metabolism of Proteins, Signal Transduction, Disease, Hemostasis, Immune System, Metabolism of RNA, Organelle Biogenesis and Maintenance, Autophagy, Chromatin Organization, Protein Localization, Neuronal System, Sensory perception, Muscle Contraction, Digestion and Absorption, and Circadian Clock. As also illustrated in FIG. 3A, in applications, there are certain proteins not involved in a specific pathway listed above, which are then placed together as “Other,” as indicated by section 306 in FIG. 3B. In some embodiments, depending on how the pathways are classified, there may be different types of pathways presented in a root level grid frame.

In some embodiments, depending on the number of proteins associated with a specific pathway, there may be more than one polygon in a root level grid for presenting proteins involved in a pathway. For example, as illustrated in FIG. 3A, there are three hexagons for presenting proteins included in the Developmental Biology pathway, five hexagons for presenting proteins included in the Disease pathway, five hexagons for presenting proteins included in the Metabolism pathway, etc. In some embodiments, when there are multiple polygons utilized for one pathway, these polygons may be clustered together to form a single section. In this single section, the borders shared between the polygons within the section may be not shown, so the section only displays the borders with the neighbor sections, or may be shown with a different color or light intensity when compared to polygon borders between different sections so that a section can be easily identified. For example, section 304 for the Metabolism pathway in FIG. 3A includes five hexagons, in which the hexagon borders within the section are not displayed. Accordingly, a shape for an overall section may be not exactly a hexagon shape but rather a shape of hexagon clusters if there is more than one hexagon for a section.

In some embodiments, when being displayed as a user interface, a section of a grid can be selected and highlighted once selected (e.g., clicked or hovered over by a mouse or by a finger or pen in a touchscreen). In some embodiments, the relevant information for the section may be further presented when the section is selected. For example, as shown in FIG. 3A, when section 304 for the Metabolism pathway is selected, the information for the section such as “Reactome pathway Metabolism (Level 0)” is also illustrated. In another example, as shown in FIG. 3B, when section 306 for “Other” is selected, the selection is highlighted, and the information for the section such as “Proteins missing from Reactome” is also illustrated. The displayed information indicates that this section includes proteins missing from the Reactome pathways (e.g., not included in any Reactome pathway).

It is to be understood that while hexagons are used for presenting each section in FIG. 3A, the present disclosure is not limited to such configuration. In some embodiments, other different shapes can be also utilized for building a map for presenting the protein expression, which includes but is not limited to triangle, rectangular, square, or other proper shapes that can be clustered together to form a map format. In some embodiments, when these other different shapes are used, the built map can be also referred to as a triangle-map or other similar names, a rectangular map or other similar names, and so on.

Referring back to FIG. 2, after the generation of the root level pathway sections in a grid frame, the process may continue with a sub-level section (or simply subsection as described earlier) generation 220, to generate certain sub-level sections (also referred to as “level 1” sections) for each pathway. Specifically, in some embodiments, one section can be further divided into certain subsections. These different subsections can be divided by a line (or by other different means), where each subsection may include a subset of proteins included in a pathway (which can be a level 1 pathway, a child of a level 0 pathway) that can be grouped together. For example, as illustrated in FIG. 3C, section 308 for “Metabolism of RNA” can be divided into five subsections, as indicated by five straight lines in section 308. Each subsection includes a subset of proteins that share certain common features or functions (e.g., in a same level 1 pathway). In one example, subsection 310 includes proteins involved in mRNA capping process in the Metabolism of RNA pathway.

In some embodiments, when a subsection is presented in a user interface and is selected, the sub-selection can be also highlighted and the corresponding information can be also displayed. For example, when subsection 310 is selected, subsection 310 is highlighted, and the information “Reactome pathway mRNA Capping (Level 1)” for the subsection is displayed, as shown in FIG. 3C. Here, “Level 1” indicates that the selected section is a subsection, and “mRNA capping” indicates that the proteins included in the subsection are related to mRNA capping in the Metabolism of RNA pathway.

In some embodiments, the area occupied by each subsection is determined based on the number of proteins or the ratio of the proteins in the subsection when compared to the total proteins included in the whole section. In some embodiments, the relative sizes of those subsections are calculated as the number of assays divided by 10 and rounded up. In general, the larger the number of proteins or the bigger the ratio, the larger the area for a subsection.

In some embodiments, not all sections in a grid frame include subsections. For example, in the illustrated embodiment in FIG. 3C, three sections corresponding to the Cellular Response to Stimuli, Chromatin Organization, Digestion and Absorption pathways do not have any subsections. This can be due to the limited number of proteins included in each section or can be due to the fact that the proteins included in each section do not share certain common features that allow grouping these proteins into subsections. As also illustrated in FIG. 3C, the section “Other” that includes proteins “missing from Reactome” does not have a subsection either, although there are a lot of proteins in the section. This is because there is no basis in the available information for grouping the proteins in subsections.

It is to be understood that while only level 0 and level 1 are illustrated in FIGS. 2 and 3C, in some embodiments, a grid may have more than two levels. For example, a sub-pathway can be further divided into sub-sub-pathways, and so on. Accordingly, there may be certain sub-subsections in a generated grid. It is also to be understood that there may be certain “Other” subsections or “Other” sub-subsections that cannot be grouped into a sub-pathway or sub-sub-pathway.

Referring back to FIG. 2, after determining the sections and subsections for biological pathways, proteins included in these sections/subsections are then assigned to specific locations in each section/subsection at step 230. After the assignment, these proteins can be then presented at the assigned locations as points, thereby forming a map structure or a grid at a map format.

In some embodiments, the assignment of each protein to a specific location within a section/subsection is random. For example, proteins included in a section/subsection can be assigned to a random location in a section/subsection, as long as the assigned locations are evenly distributed (or approximately evenly distributed) within a section/subsection.

In some embodiments, the assignment of each protein to a location within a section/subsection can be based on a predefined format, e.g., the order of the proteins involved in a pathway. For example, for proteins involved in “mRNA Capping” in section 310, these proteins can be arranged in subsection 310 based on the order that each protein is involved in the mRNA capping process. A protein that participates in the mRNA capping process earlier may be assigned to a top left location in subsection 310, the following protein that participates in the process in the next is then assigned to a next location (e.g., top right), and so on. In some embodiments, when the order of these proteins is determined, these proteins can be arranged in the section/subsection from the top to bottom and/or from the left to right or another different order according to the predefined format. In some embodiments, for the proteins included in “Other” section, these proteins may be randomly assigned, since these proteins may not participate a same process and/or pathway.

In some embodiments, there are additional means to assign proteins to specific locations in a section/subsection. For example, when assigning proteins to specific locations in a section or subsection, the proteins that are more likely upregulated or downregulated (e.g., based on a data analysis of multi-protein signatures of a large variety of cancers or other diseases) are evenly distributed (or approximately evenly distributed), so that the corresponding points with relatively large sizes are not so crowded in a specific area(s) under certain circumstances.

In some embodiments, once the locations of all proteins are assigned in each section/subsection, the assigned locations of these proteins will not change, so that one generated grid can be comparable to another, facilitating protein expression analysis and comparison.

Referring back to FIG. 2, after the locations for the points are assigned for the corresponding proteins, the protein expression levels of these proteins can be integrated into the points at step 240. This may include the adjustment of the appearance including the size and color of each point based on the relative expression level detected for each protein. For example, an upregulated protein that has a higher relative expression level (e.g., when compared to control cohort of healthy individual(s) or individuals with different diseases or at different disease stages) may have a larger point size as compared to a protein that has a relative expression level closer to the control cohort. Similarly, a downregulated protein that has a lower relative expression level (e.g., when compared to control cohort of healthy individual(s) or individuals with different diseases or at different disease stages) may have a larger point size as compared to a protein that has a relative expression level closer to the control cohort.

In some embodiments, to differentiate between upregulated proteins and downregulated proteins in a grid, different colors may be assigned to points associated with the upregulated or downregulated proteins in a first cohort relative a second (control) cohort. In one example, a red color is assigned to points associated with the upregulated proteins, and a blue color is assigned to points associated with the downregulated proteins. In some embodiments, other different color schemes can be used for the purpose, as long as the two kinds of proteins can be differentiated by color in a grid.

In some embodiments, the color intensity of each point can be further adjusted. For example, while proteins with an adjusted p-value above 0.05 are shown as small grey points, for all other proteins, the size and color intensity increase with the absolute value of the ANOVA estimate, up to a cap of ±2. In some embodiments, the scaling between (absolute) ANOVA estimate and point size and color intensity is not linear, to intentionally highlight the big associations. This scaling may also be identical for all grids produced for similar types of disease, such as all cancer types, which makes the grids entirely comparable to each other.

FIG. 3D shows an example grid with adjusted size and color for specific points in sections/subsections. As can be seen in the figure, there are different sections for different biological pathways. Among these different sections, a number of sections include subsections. In each section or subsection, there are a set of points that have different color and size. Here, the size of a point corresponds to a relative protein expression level, as compared to control cohort. The color of a point corresponds to an upregulated or downregulated expression.

As can be seen in FIG. 3D, among the sections or subsections illustrated in the figure, there are some tiny points that do not show one of the two colors as other points with a larger size. These tiny points correspond to proteins that do not have upregulated or downregulated expression. For example, p-values for these tiny points may be larger than a predefined value (e.g., 0.05 or another value), which suggests the proteins associated with these tiny points do not have a statistically significant upregulation or downregulation when compared to the control cohort.

In some embodiments, to allow the grid to be elaborated on the proteins that have abnormal protein expression levels, these tiny points can be configured not to show up in a built grid. For example, a user interface for presenting a grid may be configured to include a toggle that can be activated so as not to show these tiny points corresponding to proteins that are not statistically significantly upregulated or downregulated (e.g., the corresponding p-values are above 0.05).

FIG. 3E shows an example grid with removal of non-significant proteins (e.g., proteins that are not statistically significantly upregulated or downregulated). As illustrated, compared to FIG. 3D, the tiny points are removed from the generated grid. Accordingly, in the grid, only significantly upregulated or downregulated proteins are presented.

In some embodiments, a grid generated according to the above-described process in FIG. 2 may additionally include certain information associated with each protein presented in the grid. For example, by pointing to a specific point presented in a built grid, additional information related to the protein associated with the point may show up.

FIG. 3F shows an example grid with certain information displayed for a protein included in a grid. According to the illustrated embodiment in FIG. 3F, the display information may include, but is not limited to, protein name (e.g., insulin-like growth factor-binding protein 2 (IGFBP2)), associated assay category (e.g., assays for proteins relevant to cardiometabolic health), identification number (e.g., a UniProt ID number and/or a grid system ID), relative protein expression level (e.g., +0.55 NPX), and adjusted p-value (e.g., 9.5e-05). In some embodiments, the sample information used for protein expression detection is also presented. For example, as illustrated in FIG. 3F, the protein information is acquired based on a comparison of 51 acute myeloid leukemia patients with 1842 patients with other types of cancers.

It is to be understood that, in humans or other animals, one single protein may be involved in different biological pathways. In addition, even within a single pathway, one protein may be involved in different processes or functional activities. Accordingly, in a grid generated based on the above process 200, one protein may be included in different sections and/or different subsections.

FIG. 3G illustrates an example grid with a single protein included in multiple sections/subsections. As illustrated in FIG. 3G, nitric oxide synthase 3 (NOS3) is a protein that can be involved in a Metabolism pathway, Signal Transduction pathway, or Hemostasis pathway. Accordingly, when the NOS3 protein is selected from one of these pathways, the other involved pathways for the protein are also displayed. As also illustrated in FIG. 3G, within the Metabolism pathway, the NOS3 protein can be involved in different processes (e.g., level-1 pathways) or classified into functional groups of the Metabolism pathway, and thus can be included in different subsections of the pathway.

It is to be understood that, when a same protein is displayed in different sections/subsections of a grid, the points corresponding to the protein will have the same size and color within the same grid, as illustrated in FIG. 3G. However, in different grids that are generated based on the protein expression levels detected from different samples, the size and color between these grids may be different. Also, grids for different cancers may show different sizes and/or colors for a same protein.

FIGS. 4A-4N illustrates various grids generated based on the differential protein expression levels detected from different types of cancer patient samples. Specifically, FIG. 4A illustrates a grid generated based on the samples collected from acute myeloid leukemia patients when compared to other cancer patients, FIG. 4B illustrates a grid generated based on the samples collected from chronic lymphocytic leukemia patients when compared to other cancer patients, FIG. 4C illustrates a grid generated based on the samples collected from lymphoma patients when compared to other cancer patients, FIG. 4D illustrates a grid generated based on the samples collected from myeloma patients when compared to other cancer patients, FIG. 4E illustrates a grid generated based on the samples collected from breast cancer patients when compared to other cancer patients, FIG. 4F illustrates a grid generated based on the samples collected from cervical cancer patients when compared to other cancer patients, FIG. 4G illustrates a grid generated based on the samples collected from endometrial cancer patients when compared to other cancer patients, FIG. 4H illustrates a grid generated based on the samples collected from glioma patients when compared to other cancer patients, FIG. 4I illustrates a grid generated based on the samples collected from lung cancer patients when compared to other cancer patients, FIG. 4J illustrates a grid generated based on the samples collected from meningioma patients when compared to other cancer patients, FIG. 4K illustrates a grid generated based on the samples collected from neuroendocrine cancer patients when compared to other cancer patients, FIG. 4L illustrates a grid generated based on the samples collected from ovarian cancer patients when compared to other cancer patients, FIG. 4M illustrates a grid generated based on the samples collected from pituitary cancer patients when compared to other cancer patients, and FIG. 4N illustrates a grid generated based on the samples collected from prostate cancer patients when compared to other cancer patients.

In the FIGS. 4A-4N, only points corresponding to proteins with a p-value less than 0.05 (e.g., only significantly upregulated or downregulated proteins) are illustrated in each grid. As can be seen from the grids illustrated in FIGS. 4A-4N, the abnormal protein expression levels in different types of cancer patients show a clear difference, which can be demonstrated by the occurrence of the different points, the size of each displayed point, and/or the color of each displayed point in different grids. In some embodiments, the displayed point locations, point sizes, and point colors in a grid can form a unique pattern for a specific type of cancer, due to the unique protein expression levels among patients with different types of cancer. These unique cancer-specific patterns can be then used for understanding disease, or for other different purposes as described earlier.

In some embodiments, the grids illustrated in FIGS. 4A-4N can be used to build a grid library, as described earlier. The grids in the library can be generated at the beginning stage based on a limited number of patient samples that represent different ages, sex, regions, etc. In some embodiments, if there are enough samples available, grids can be also generated for different stages of cancer. In this way, a library of grids reflecting different cancers (or other diseases according to an embodiment) and/or different stages of cancer (or other diseases) can be generated. In some embodiments, based on the protein expression levels further collected during diagnosis processes or other processes, grids included in the library can be dynamically updated. For example, the detected protein expression level (e.g., NPX difference) for a protein can be dynamically adjusted if there are more representative samples available during the later processes after an initial grid is generated.

### Example Method

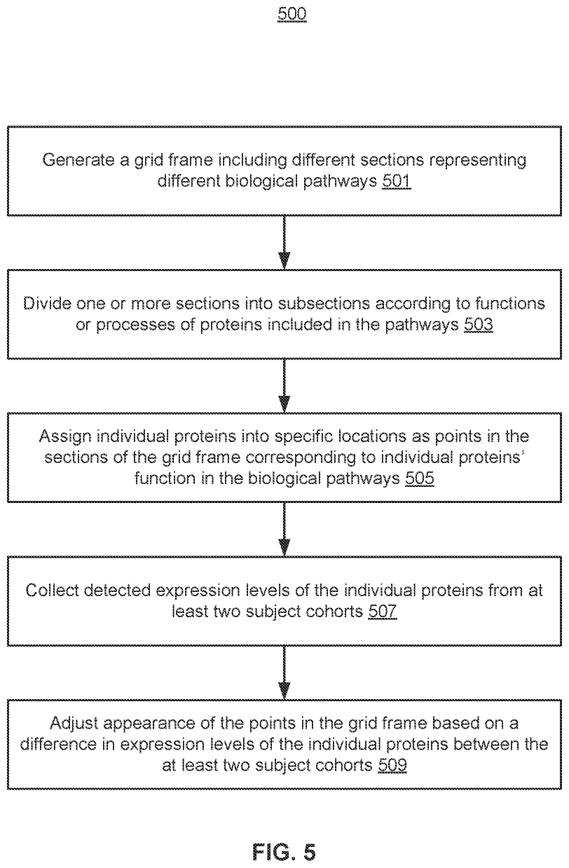

Additionally disclosed herein is an example method 500 for building a grid, according to an embodiment. In some embodiments, method 500 may be performed by various components of the disease examination system 130 (e.g., by the grid generation module 160). In some embodiments, method 500 may include steps 501-509. It is to be understood that some of the steps may be optional. Further, some of the steps may be performed simultaneously, or in a different order than that shown in FIG. 5.

In Step 501, a grid frame including different sections representing different biological pathways is first generated. The generated grid frame includes a plurality of contiguous polygons clustered together to form a single map-format grid frame. Each section in the frame may correspond to one pathway. Depending on the number of proteins included in a biological pathway, each section in the grid frame may include one or more polygons. In one example, the number of polygons assigned to a pathway is the number of assays mapped to that pathway divided by 50, rounded up. When there is more than one polygon included in a section, the border(s) between the polygons within a same section may be displayed in a lighter and/or thinner line or totally removed.

In Step 503, one or more sections are further divided into subsections according to the functions or processes (e.g., level-1 pathways) of the proteins included in the corresponding pathways. For example, for proteins involved in a Protein Localization pathway, these proteins may be divided into four subsections based on their functions: proteins for mitochondrial protein import, proteins for peroxisomal protein import, proteins for peroxisomal membrane protein import, and proteins for insertion of tail-anchored proteins into the endoplasmic reticulum membrane. According to an embodiment, one section can be divided into any number (e.g., 2, 3, 4, 5, 6, 7, 8, 9, 10, . . . ) of subsections. For example, in one configuration, a section is divided into three subsections. In another configuration, a same section can be divided into four sections. In some embodiments, once subsections are configured for each section, the configuration will remain unchanged for easier grid-to-grid comparison. In some embodiments, a section is not divided into any subsections. For example, there is no subsection for the “Chromatin Organization” section, according to an embodiment.

In Step 505, proteins included in each section or subsection are assigned to specific locations in the grid frame as points. The assignment of protein locations within a section/subsection can be randomly or based on a predefined format (e.g., based on a sequential order of the proteins involved in the process(es) include in the corresponding pathway). In some embodiments, once assigned, the point locations associated with specific proteins within a section/subsection will not change. This allows comparing the protein expression level of a protein in different samples by checking the point size and color in a same location(s) of different grids.

In Step 507, protein expression levels of each protein included in the grid are collected from at least two subject cohorts. In some embodiments, the protein expression levels of the proteins included in the grid can be detected by using a platform configured for proteomic analysis, which may allow the detection of expression levels of all proteins included in the grid. In addition, the at least two subject cohorts may include a first subject cohort that includes a group of patients with a same type/state of cancer, and a second subject cohort that includes a group of health individuals or a group of patients with other types of cancer. It is to be understood, the protein expression levels can be collected at any moment before generating a grid or during the process of generating a grid.

In Step 509, the appearance including size and color of points in the sections/subsections are adjusted based on a difference in expression levels of the individual proteins between the at least two subject cohorts. The more obvious change of a protein expression (e.g., by comparing protein expression levels of the first subject cohort with those of the second subject cohort), the larger size of the corresponding point. In addition, different colors can be used to indicate whether a protein is upregulated or downregulated. In some embodiments, there is no size and/or color change for a point if the corresponding protein does not show obvious expression change (e.g., when compared to healthy individuals). In some embodiments, once the size and color are properly determined for each point, a grid is then generated.

In some embodiments, the generated grid can be displayed in a user interface that allows a user to interact with the grid. For example, when a user selects a section or subsection, the section or subsection frame can be highlighted when compared to other unselected sections or subsections. In some embodiments, relevant information for a selected section or subsection can be automatically popped up, to allow a user to check the information related to the selected section or subsection. Similarly, when a point is selected, relevant information for a corresponding protein can be also automatically popped up. In some embodiments, if the selected protein is involved in multiple sections or subsections, the corresponding points can be also automatically highlighted, to facilitate the understanding of the proteins in different pathways or different processes in a same pathway.

### Non-Transitory Computer Readable Medium

Also provided herein is a computer-readable medium comprising computer-executable instructions configured to implement any of the methods described herein. In various embodiments, the computer-readable medium is a non-transitory computer-readable medium. In some embodiments, the computer-readable medium is a part of a computer system (e.g., a memory of a computer system).

### Computing Device

The methods described above, including the methods of training and deploying machine learning models are, in some embodiments, performed on a computing device. Examples of a computing device can include a personal computer, desktop computer laptop, server computer, a computing node within a cluster, message processors, hand-held devices, multi-processor systems, microprocessor-based or programmable consumer electronics, network PCs, minicomputers, mainframe computers, mobile telephones, PDAs, tablets, pagers, routers, switches, and the like.

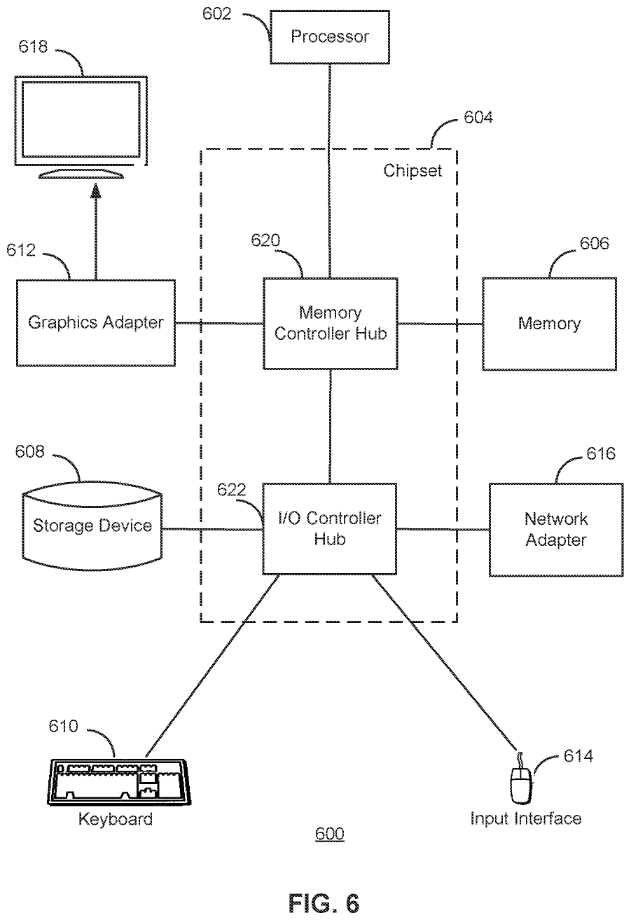

FIG. 6 illustrates an example computing device for implementing the systems and methods described above. In some embodiments, the computing device 600 shown in FIG. 6 includes at least one processor 602 coupled to a chipset 604. The chipset 604 includes a memory controller hub 620 and an input/output (I/O) controller hub 622. A memory 606 and a graphics adapter 612 are coupled to the memory controller hub 620, and a display 618 is coupled to the graphics adapter 612. A storage device 608, an input interface 614, and network adapter 616 are coupled to the I/O controller hub 622. Other embodiments of the computing device 600 have different architectures.

The storage device 608 is a non-transitory computer-readable storage medium such as a hard drive, compact disk read-only memory (CD-ROM), DVD, or a solid-state memory device. Memory 606 holds instructions and data used by processor 602. The input interface 614 is a touch-screen interface, a mouse, trackball, or other types of input interface, a keyboard 610, or some combination thereof, and is used to input data into the computing device 600. In some embodiments, the computing device 600 may be configured to receive input (e.g., commands) from the input interface 614 via gestures from the user. The graphics adapter 612 displays images, graphs, and other information on the display 618. The network adapter 616 couples the computing device 600 to one or more computer networks.

The computing device 600 is adapted to execute computer program modules for providing the functionality described herein. As used herein, the term “module” refers to computer program logic used to provide the specified functionality. Thus, a module can be implemented in hardware, firmware, and/or software. In one embodiment, program modules are stored on the storage device 608, loaded into memory 606, and executed by processor 602.

The types of computing devices 600 can vary from the embodiments described herein. For example, the computing device 600 can lack some of the components described above, such as graphics adapters 612, input interface 614, and displays 618. In some embodiments, a computing device 600 can include a processor 602 for executing instructions stored on a memory 606.

The methods for generating grids can, in various embodiments, be implemented in hardware or software, or a combination of both. In one embodiment, a non-transitory machine-readable storage medium, such as one described above, is provided, the medium comprising a data storage material encoded with machine-readable data which, when using a machine programmed with instructions for using said data, is capable of displaying any of the datasets and execution and results of any machine learning model of this disclosure. Such data can be used for a variety of purposes, such as examination and understanding of disease mechanisms, and the like. Embodiments of the methods described above can be implemented in computer programs executing on programmable computers, comprising a processor, a data storage system (including volatile and non-volatile memory and/or storage elements), a graphics adapter, an input interface, a network adapter, at least one input device, and at least one output device. A display is coupled to the graphics adapter. Program code is applied to input data to perform the functions described above and generate output information. The output information is applied to one or more output devices, in a known fashion. The computer can be, for example, a personal computer, microcomputer, or workstation of conventional design.

Each program can be implemented in a high-level procedural or object-oriented programming language to communicate with a computer system. However, the programs can be implemented in assembly or machine language, if desired. In any case, the language can be a compiled or interpreted language. Each such computer program is preferably stored on a storage media or device (e.g., ROM or magnetic diskette) readable by a general or special-purpose programmable computer, for configuring and operating the computer when the storage media or device is read by the computer to perform the procedures described herein. The system can also be considered to be implemented as a computer-readable storage medium, configured with a computer program, where the storage medium so configured causes a computer to operate in a specific and predefined manner to perform the functions described herein.

The signature patterns and databases thereof can be provided in a variety of media to facilitate their use. “Media” refers to a manufacturer that is capable of recording and reproducing the signature pattern information of the present disclosure. The databases of the present disclosure can be recorded on computer-readable media (e.g., any medium that can be read and accessed directly by a computer). Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skills in the art can readily appreciate how any of the presently known computer readable mediums can be used to create a manufacture comprising a recording of the present database information. “Recorded” refers to a process for storing information on computer-readable medium, using any such methods as known in the art. Any convenient data storage structure can be chosen, based on the means used to access the stored information. A variety of data processor programs and formats can be used for storage (e.g., word processing text file, database format, etc.).

### EXAMPLE APPLICATIONS

### Example 1: Disease Atlas-Cancer

Cancer is one of our major global health issues, accounting for around 10 million deaths annually. Despite substantial improvements in cancer therapy over recent decades, there is still significant scope for improved diagnostics and treatment. Precision and personalized medicine, fueled by advancements in proteomics, has the potential to contribute to this process. Of specific interest to cancer precision medicine is early and accurate diagnosis since this can dramatically improve the prognosis of many types of cancers.

The proximity extension assay (PEA) technology (Olink Proteomics AB, Uppsala, Sweden) is well suited to support advancements in precision cancer medicine. It allows for the simultaneous quantification of several thousand proteins with high specificity and sensitivity from just a few microliters of a biological sample. This paves the way for proteomic profiling of various cancer sub-types, creating for each a map of its underlying proteome and biological context.

The data analyzed and presented in Example 1 were generated from a pan-cancer cohort within a biobank covering several important and prevalent cancer types including but not limited to, breast, prostate, and lung cancer. Plasma samples were collected from more than 1,500 cancer patients around the time of diagnosis and were subsequently analyzed on a proteomics analysis platform. Rather than utilizing a control group of healthy individuals, each cancer type was compared to the other cancers.

Protein measurements were presented as NPX (Normalized Protein expression) values, which are relative and can be compared within, but not across, assays. A difference of 1 NPX between two measurements for the same protein assay represents approximately a doubling of the protein concentration.

### Cohort Description

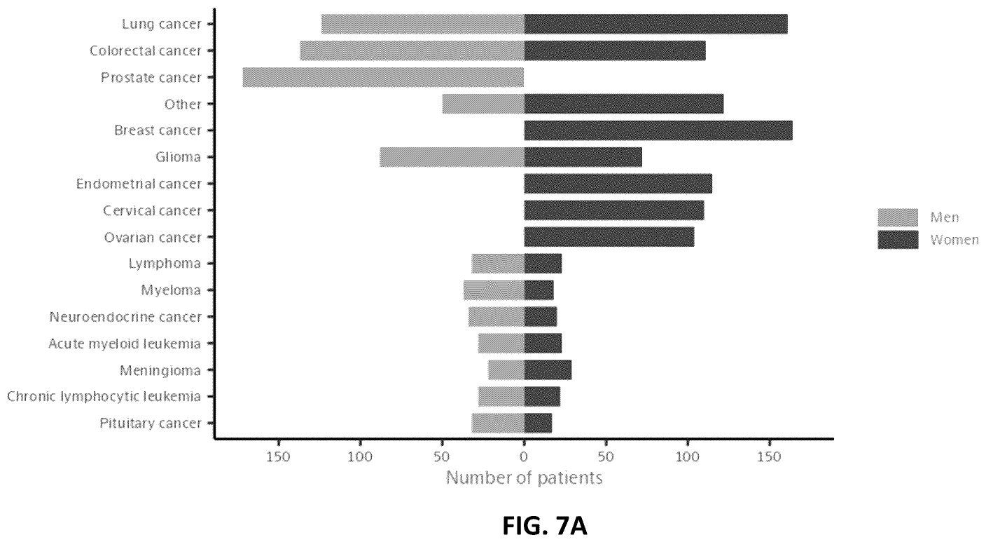

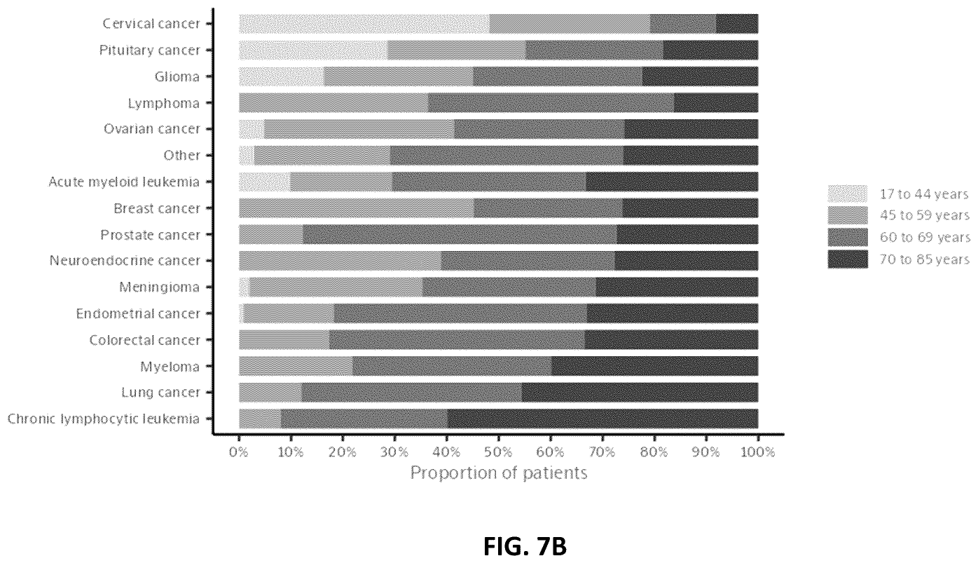

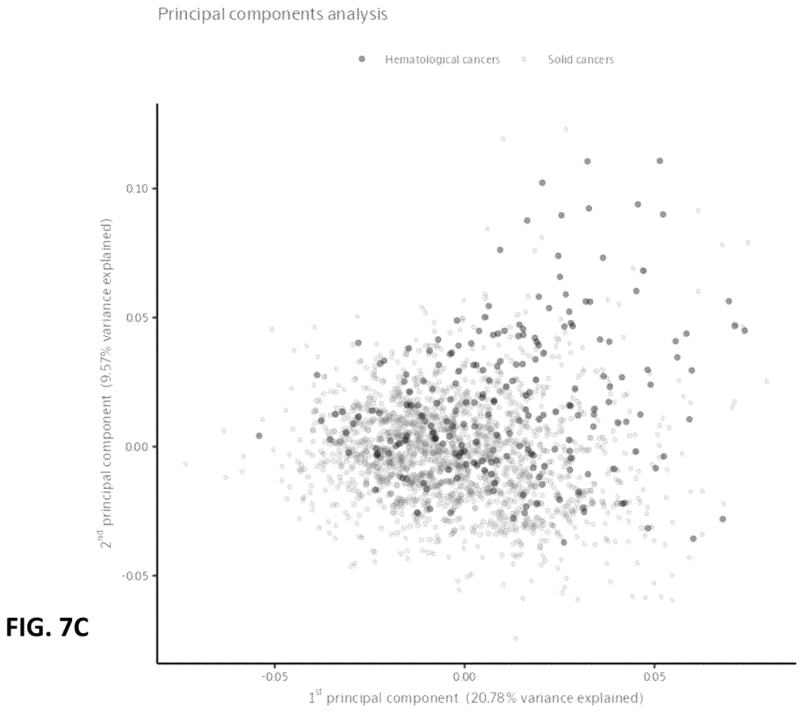

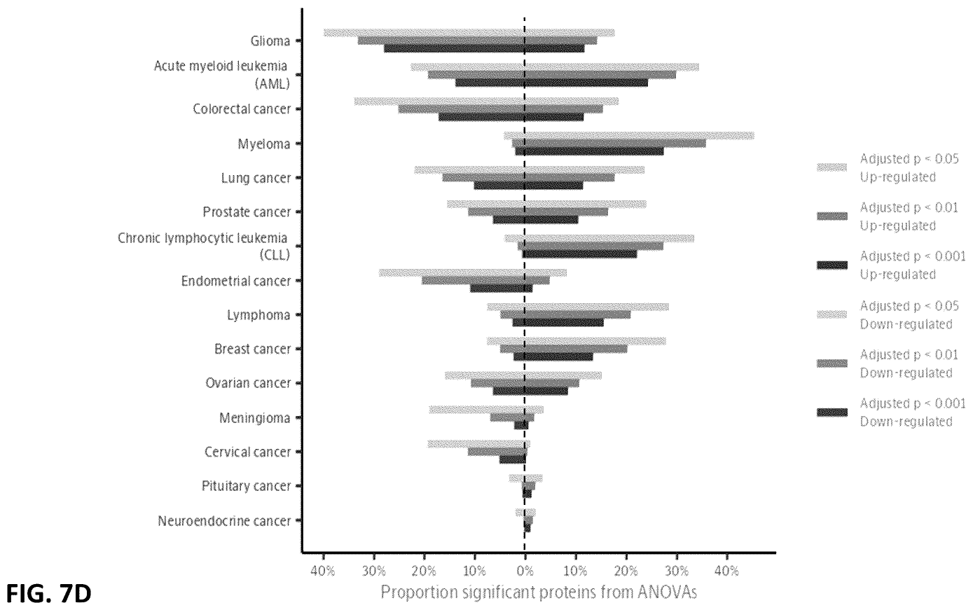

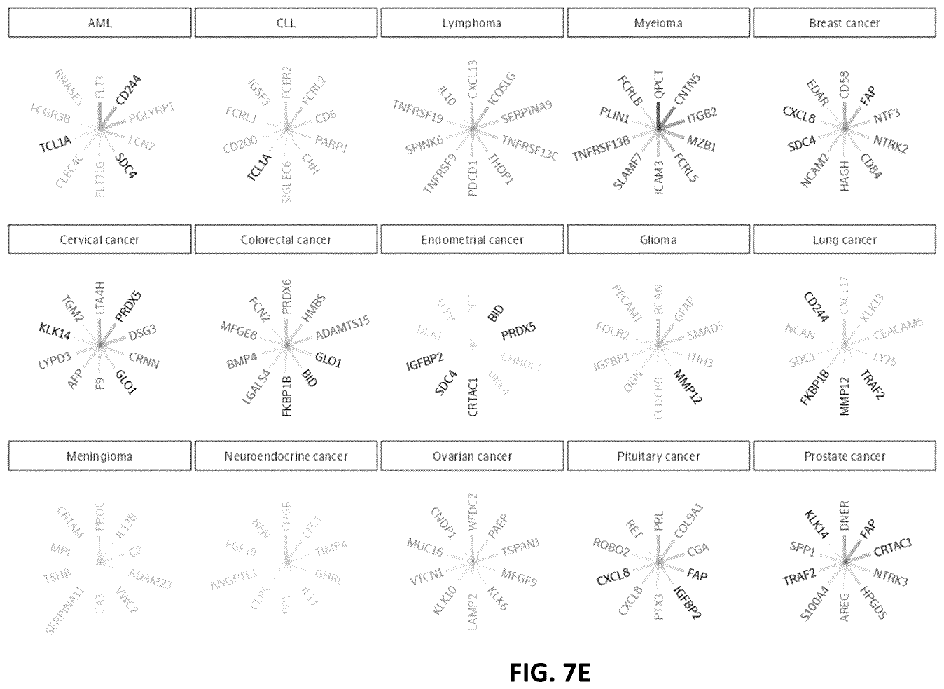

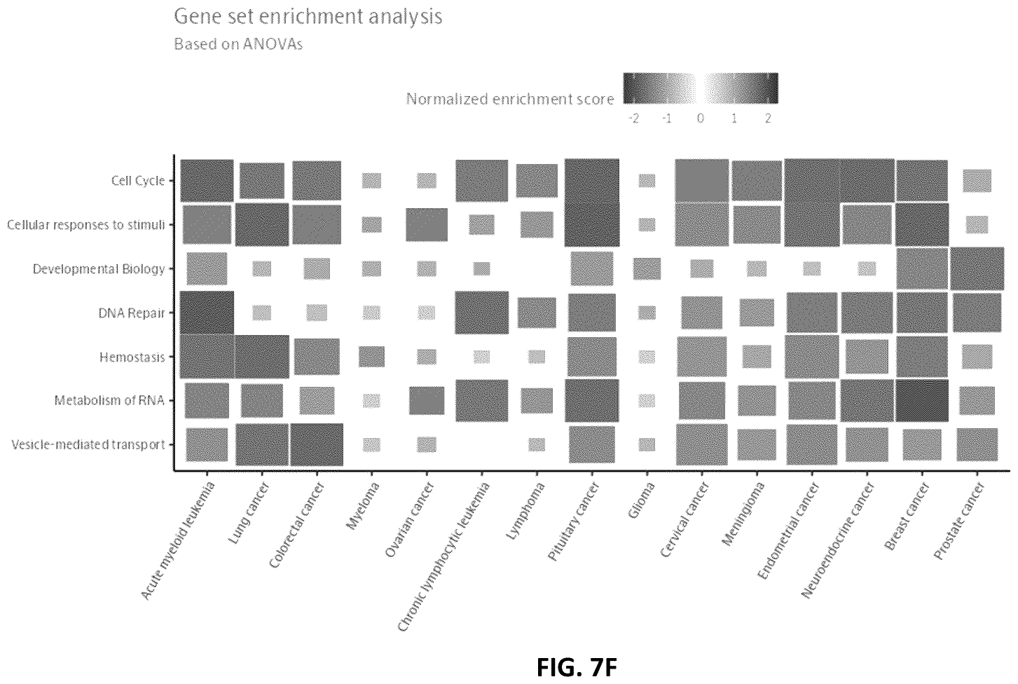

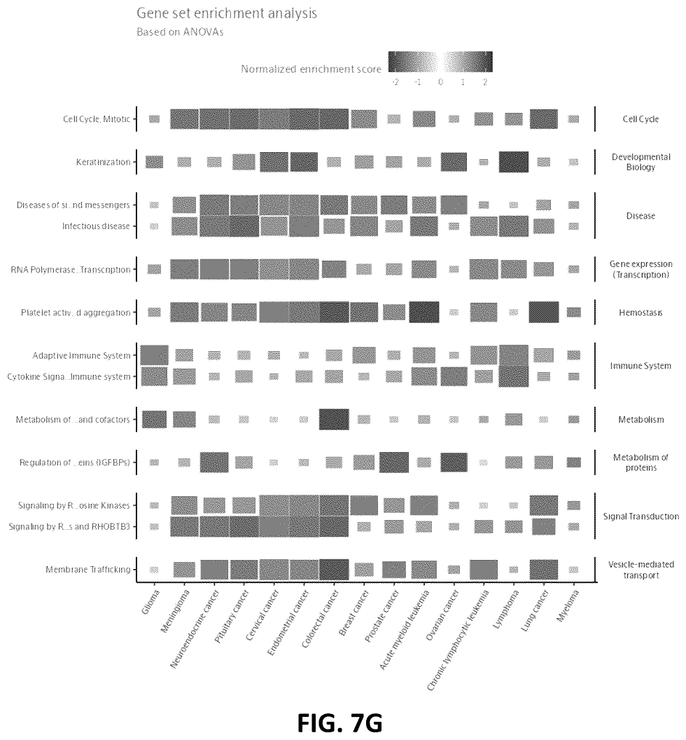

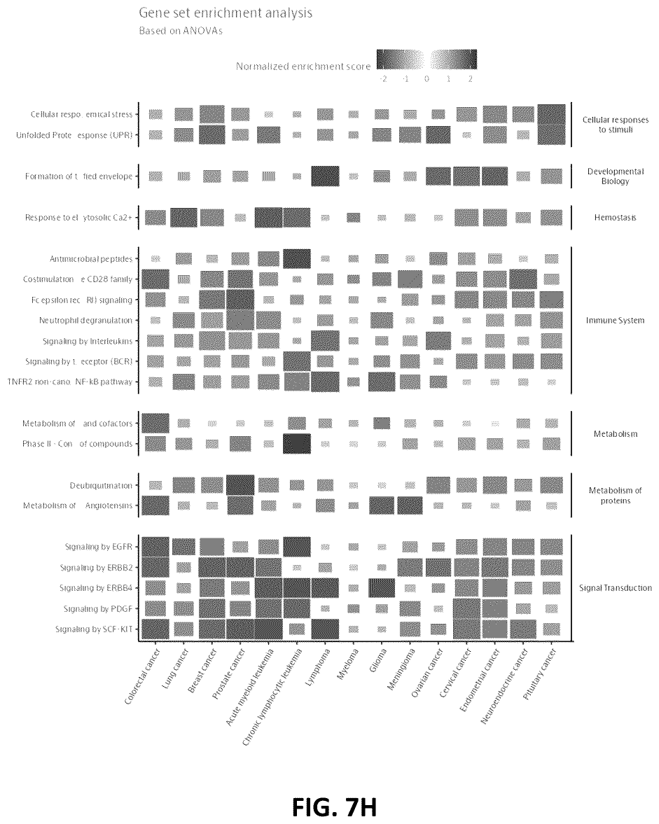

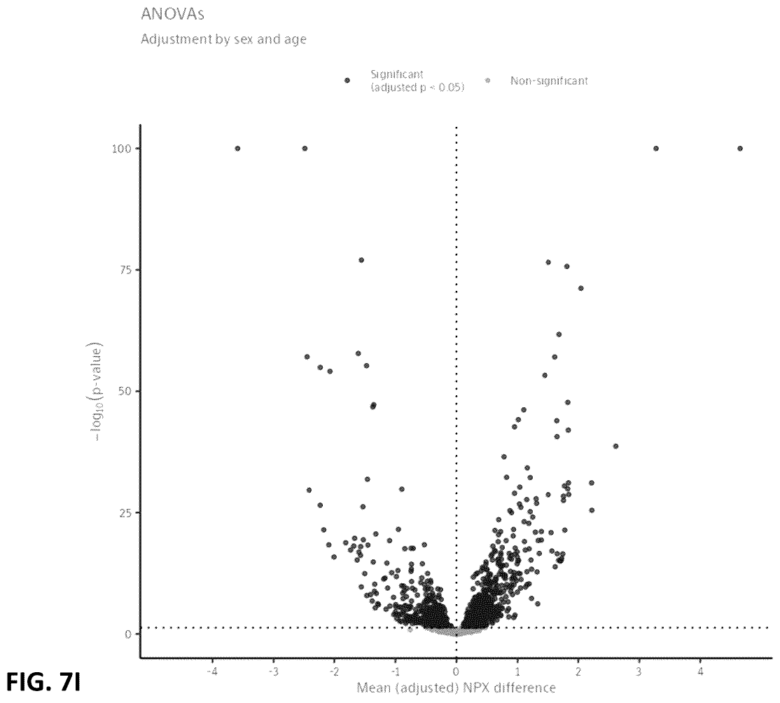

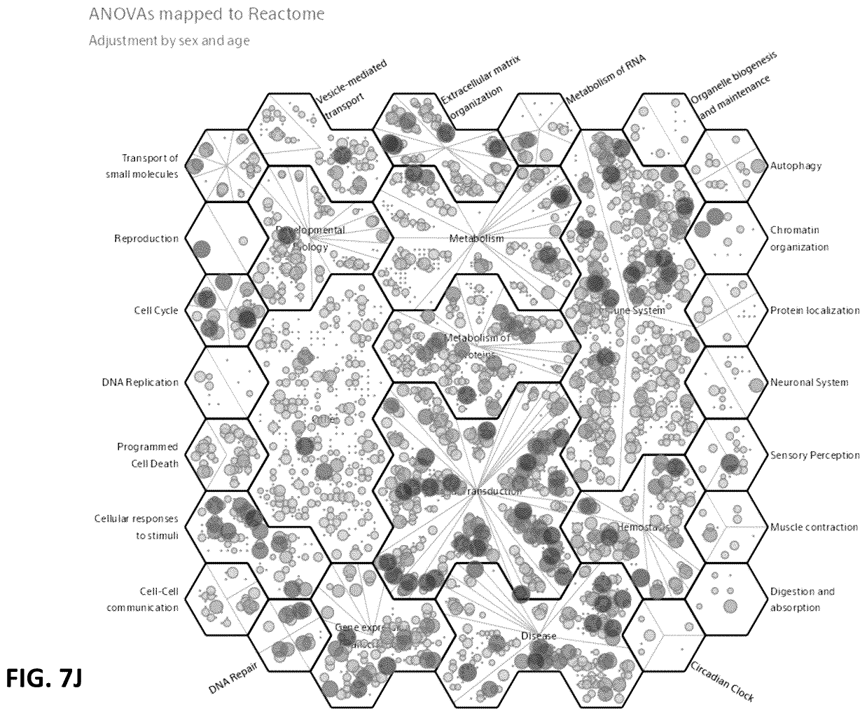

The pan-cancer cohort used in Example 1 comprises 1,895 patients, of whom 1,111 are women and 784 are men. All patients of the pan-cancer cohort were taken from the U-CAN biobank (4), with blood sampled from consenting patients as part of their routine care. Only patients without any known concurrent or previous cancer diagnosis were included in the study. One sample was taken per patient around the time of diagnosis, before commencing any cancer treatment. The resulting protein assay data were combined with relevant patient-level clinical and demographic data. Of the patients, 1,723 belong to one of 15 cancer types of primary interest, while the rest are grouped as Other. The number of patients per cancer type, broken down by patient sex, is provided in Table 1. It can also be illustrated in a diagram, such as FIG. 7A. The most frequent cancer type is lung cancer with 285 patients, whereas pituitary cancer, with 49 patients, is the least frequent. The age range across all patients is 17 to 85 years, with a mean of 63.0 years. Overall, patients diagnosed with cervical cancer are the youngest, while chronic lymphocytic leukemia patients are the oldest. It is to be understood that for data privacy reasons, patient age has been grouped into intervals. The age distribution for each cancer type is shown in Table 2 and can be illustrated in a diagram, such as FIG. 7B.

TABLE 1
underlying FIG. 7A

| Cancer type | Women | Men |
|---|---|---|
| Lung cancer | 161 | 124 |
| Colorectal cancer | 111 | 137 |
| Prostate cancer | 0 | 172 |
| Other | 122 | 50 |
| Breast cancer | 164 | 0 |
| Glioma | 72 | 88 |
| Endometrial cancer | 115 | 0 |
| Cervical cancer | 110 | 0 |
| Ovarian cancer | 104 | 0 |
| Lymphoma | 23 | 32 |
| Myeloma | 18 | 37 |
| Neuroendocrine cancer | 20 | 34 |
| Acute myeloid leukemia | 23 | 28 |
| Meningioma | 29 | 22 |
| Chronic lymphocytic leukemia | 22 | 28 |
| Pituitary cancer | 17 | 32 |

TABLE 2
FIG. 7B

| | 17-44 | 45-59 | 60-69 | 70-85 |
|---|---|---|---|---|
| Cancer type | years | years | years | years |
| Lung cancer | 0 | 34 | 121 | 130 |
| Colorectal cancer | 0 | 43 | 122 | 83 |
| Prostate cancer | 0 | 21 | 104 | 47 |
| Other | 5 | 45 | 77 | 45 |
| Breast cancer | 0 | 74 | 47 | 43 |
| Glioma | 26 | 46 | 52 | 36 |
| Endometrial cancer | 1 | 20 | 56 | 38 |
| Cervical cancer | 53 | 34 | 14 | 9 |
| Ovarian cancer | 5 | 38 | 34 | 27 |
| Lymphoma | 0 | 20 | 26 | 9 |
| Myeloma | 0 | 12 | 21 | 22 |
| Neuroendocrine | 0 | 21 | 18 | 15 |
| cancer | | | | |
| Acute myeloid | 5 | 10 | 19 | 17 |
| leukemia | | | | |
| Meningioma | 1 | 17 | 17 | 16 |
| Chronic lymphocytic | 0 | 4 | 16 | 30 |
| leukemia | | | | |
| Pituitary cancer | 14 | 13 | 13 | 9 |

### Pan-Cancer Proteomics

The results for all the cancer types from Example I are further illustrated. All data points flagged with Quality control (QC) warnings were excluded and one poorly performing assay was entirely excluded. The analyzed data set contains a total of 1,471 protein assays for 1,462 unique proteins. Each assay is identified by a unique ID, corresponding to a unique combination of protein and panel.

To get a high-level overview of the proteomic expression among all samples, a principal components analysis (PCA) was performed for all NPX measurements. Data points removed due to QC warnings were replaced by the corresponding assays' median NPX values. The resulting projection of all samples onto the two main principal components can be plotted into a diagram, such as illustrated in FIG. 7C. There is a slight gradient of difference in protein levels between hematological and solid cancers from the bottom-left to the top-right of the plot. (The hematological cancers are acute myeloid leukemia, chronic lymphocytic leukemia, lymphoma, and myeloma.) Hover over individual points in the plot diagram allows to reveal their cancer type, or hover over the legend to identify the corresponding data points in the plot. Using a toggle (not shown) also allows to color samples by individual cancers.

To identify proteins that differ in their NPX levels between cancers, standard differential expression analyses were performed for each cancer type in turn, by fitting one ANOVA (analysis of variance) model per protein. For each cancer type, the protein levels (i.e., the NPX values) between the group of patients with that cancer and the group consisting of all other patients were compared, excluding men for women-only diagnoses and vice versa. The analyses were adjusted for age and, when applicable, sex, and the estimated NPX differences as well as associated p-values were extracted. Finally, for each cancer type, all p-values were adjusted for multiple testing. A summary of all cancer types can be illustrated in a figure, such as FIG. 7D. Glioma has the most differentially expressed proteins in total, whereas myeloma ranks highest in terms of up-regulated proteins specifically.

In addition to the single-protein differential expression analysis just described, multi-protein modeling using lasso logistic regression was also performed. Two models were fit per cancer type, one using all proteins as predictors, and one using only up-regulated proteins. As previously described, one cancer was compared to all others, including age and sex (where applicable) as additional covariates, and restricted the analysis to only women or only men for single-sex cancers. Prior to model fitting, data points removed due to QC warnings were replaced by their corresponding median NPX values.

A lasso is a form of shrinkage regression, where models can be made arbitrarily small by increasing the shrinkage. Each cancer model was tuned to contain precisely ten proteins, aiming for a small set of strong predictors. However, these models may not necessarily be optimal in terms of predictive performance.

The ten selected proteins for each cancer type can be visualized in a figure such as FIG. 7E. The proteins are ordered clockwise by the absolute values of their coefficient estimates, with the strongest associations pointing upwards. Line color intensity is also used to indicate the strength of the association. Hovering over lines or names allows for more information to be displayed. Proteins selected for multiple cancer types are indicated by the bold black text, and hovering over this bold black text allows it to reveal all cancers for which they were selected.

To gain further insight into the biological systems and processes affected by the various cancer types, pathway enrichment of the ANOVA estimates against Reactome as provided in the Molecular signatures database (MSigDB) was further performed. Using Gene Set Enrichment Analysis (GSEA), for each cancer type, a Normalized Enrichment Score (NES) with an associated p-value for each Reactome pathway containing at least ten measured proteins was obtained. A positive NES value indicates generally high ANOVA estimates for the proteins included in the pathway, while a negative NES indicates generally low (below 0) estimates.

The results are displayed in heatmaps, such as illustrated in FIG. 7F-7H, with color indicating the NES and size inversely proportional to the adjusted p-value. Cancer types are arranged by similarity in the NES and pathways are ordered alphabetically. Only pathways with an adjusted p-value below 0.05 for any of the cancers are included, and only from one Reactome level at a time. The pathways in Reactome are arranged in a hierarchical structure, where level is used to indicate hierarchical depth: Root pathways are found on level 0 (FIG. 7F), their children on level 1 (FIG. 7G), level 2 (FIG. 7H), and so on. Hovering over the boxes in the heatmaps (FIGS. 7F-7H) allows for more information to be displayed, and the Reactome pathway level can be selected by using the drop-down menu (not shown).

In-depth results for a specific cancer type, including volcano plots, classification models, and more detailed pathway enrichment and annotation can be further explored. FIG. 7I shows an example volcano plot for ANOVA-based differential expression results for acute myeloid leukemia.

### Example Grid

FIG. 7J shows an example grid that provides a presentation of a single-protein differential expression analysis results. The grid illustrated in FIG. 7J is similar to the one shown in FIG. 3D. This visualization aims to highlight biological pathways containing proteins strongly associated with a type of cancer (e.g., acute myeloid leukemia). Each measured protein appears as a point in all Reactome level 0 and 1 pathways to which it is annotated, with proteins missing from Reactome annotated as “Other”. Consequently, the same protein can appear multiple times. The size and color intensity in the grid depend on the absolute mean adjusted NPX difference as estimated in the ANOVAs, with red and blue colors indicating higher and lower protein levels, respectively. Proteins with an adjusted p-value above 0.05 are shown as small grey points. Note that the scaling of size and color intensity is not linear with the ANOVA estimates, to intentionally highlight the big associations. Furthermore, the scaling is the same for all cancer types.

The size of each Reactome pathway in the grid is related, but not directly proportional, to the number of proteins. The pathways' locations in the grid have no direct meaning, except that pathways on level 1 are included under their respective roots on level 0. Each protein's location within a pathway is random. In FIG. 7, hovering over individual points, areas, and borders allows for detailed information to be displayed, and activating a toggle (not shown) allows to remove proteins with adjusted p-values above 0.05.

It is to be understood that while a grid usually has an identical structure for all cancer types, the size, color, and intensity of individual points vary depending on the specific ANOVA estimates for the cancer of interest.

In some embodiments, to align the grid construction with the pathway enrichment, MSigDB is used as the starting point and mapped proteins to Reactome pathways in the same way as described above for the enrichment analysis. Pathways that either do not map to any proteins or that are missing from the reference Reactome list, are not part of the grid. While Example I aims for basic alignment between annotation and enrichment, the two serve slightly different purposes and have different constraints. For example, both very large and very small pathways can pose problems for enrichment analysis, but can still be used for annotation and visual presentation. Accordingly, the following additions may be made to the grid version of Reactome compared to that used for the enrichment analysis. First, a protein mapped to a pathway in the MSigDB Reactome version is considered mapped to all true parent pathways (as defined by the reference list), even if those pathways themselves are not included in MSigDB. Importantly, the grid consequently contains many more root level pathways than the pathway enrichment docs. Second, proteins that do not map to any pathway are placed in an artificial root category called “Other”. Similarly, proteins mapped to a root pathway but not to any of its child pathways are placed in an artificial level 1 “Other” category within that root.

In a grid, each root (level 0) Reactome pathway is represented by one or several contiguous hexagons. The number of hexagons assigned to a pathway is the number of assays mapped to that pathway divided by 50, rounded up. Similarly, each root pathway is divided into sub-areas representing the level 1 pathways within that root. The relative sizes of those sub-areas are calculated as the number of assays divided by 10 and rounded up. In contrast to its size, the location of a pathway in the grid has no direct meaning except for the relationship between a root pathway and its children. All proteins mapped to a given level 1 pathway (or an “Other” category) were given a random coordinate within the pathway's area. Since a single protein may occur in several pathways, it can be represented multiple times in the grid.

For a given cancer type, each point conveys information about the corresponding ANOVA results. Proteins with an adjusted p-value above 0.05 are shown as small grey points. For all other proteins, the size and color intensity increase with the absolute value of the ANOVA estimate, up to a cap of +2. The scaling between (absolute) ANOVA estimate and point size and color intensity is not linear, to intentionally highlight the big associations. This scaling is also identical for all cancer types, which makes their grids entirely comparable to each other.

Altogether, disclosed herein is a method and system for building a grid for presenting abnormal protein expression levels. The generated grid presents a lot of proteins in a single map format, which facilitates evaluating a lot of protein expression levels without going through pages-after-pages of information.

Although the techniques have been described in language specific to structural features and/or methodological acts, it is to be understood that the subject matter defined in the appended claims is not necessarily limited to the specific features or acts described. Rather, the specific features and acts are disclosed as example forms of implementing the claimed subject matter, and other equivalent features and methods are intended to be within the scope of the appended claims. Further, various different embodiments are described and it is to be appreciated that each described embodiment can be implemented independently or in connection with one or more other described embodiments.

What is claimed is:

1. A method of building a grid for presenting differential protein expression, the method comprising:
building a grid frame including different sections representing different biological pathways;
assigning individual proteins to specific locations as points in the sections of the grid frame corresponding to individual proteins' function in the biological pathways;
collecting detected expression levels of the individual proteins from at least two subject cohorts; and
adjusting appearance of the points in the grid frame based on a difference in expression levels of the individual proteins between the at least two subject cohorts.

2. The method of claim 1, wherein the appearance of the points in the grid frame are adjusted based on a difference in the relative expression levels of the individual proteins between the at least two subject cohorts.

3. The method of claim 1, wherein the grid frame comprises a plurality of hexagons.

4. The method of claim 1, wherein generating the grid frame comprises generating a plurality of hexagons clustered together to form a single map.

5. The method of claim 3, wherein each section comprises one or more hexagons.

6. The method of claim 5, wherein each section independently comprises 1, 2, 3, 4, 5, 6, 7, 8, 9, 10, or a larger number of hexagons.

7. The method of claim 1, wherein each section corresponds to a biological pathway.

8. The method of claim 1, wherein each of one or more of the sections is divided into multiple subsections according to functions of proteins included in a corresponding biological pathway.

9. The method of claim 7, wherein each of one or more of the sections is divided into multiple subsections according to sub-pathways of the biological pathway corresponding to the section.

10. The method of claim 8, wherein the individual proteins are randomly assigned into specific locations as points in the sections or subsections.

11. The method of claim 8, wherein the individual proteins are assigned to specific locations as points in the sections or subsections according to a predefined format.

12. The method of claim 1, wherein size of the points in the grid frame is adjusted based on differences in detected normalized protein expression values for the individual proteins between the at least two subject cohorts.

13. The method of claim 1, wherein color of the points in the grid frame is adjusted based on upregulated expression or downregulated expression of the individual proteins for a subject cohort as compared to at least one other subject cohort.

14. The method of claim 13, wherein color of the points in the grid frame is adjusted to be different for upregulated expression and downregulated expression of the individual proteins, as compared between the at least two subject cohorts.

15. A method of a grid-based examination of differential protein expression, the method comprising:
collecting protein expression levels of individual proteins for at least a first and a second subject cohorts;
building a grid frame including different sections representing different biological pathways;
assigning individual proteins into specific locations as points in the sections of the grid frame corresponding to the individual proteins' function in the biological pathways;
adjusting appearance of the points in the grid frame based on a difference in expression levels of the individual proteins between the first and second subject cohorts; and
examining differential protein expression of the first and second subject cohorts based on the appearance of the points.

16. The method of claim 15, wherein the appearance of the points in the grid frame are adjusted based on a difference in the relative expression levels of the individual proteins between the at least two subject cohorts.

17. The method of claim 15, wherein one subject cohort is comprised of healthy controls.

18. A grid for presenting differential protein expression, comprising:
a plurality of sections corresponding to different biological pathways; and
one or more points in each of the plurality of sections, each of the one or more points corresponding to a specific protein, each of the one or more points having an appearance corresponding to a protein expression level of the corresponding protein in a first subject cohort relative to a protein expression level of the same protein in a second subject cohort.

19. The grid of claim 18, wherein each of the plurality of sections includes one or more hexagons, and one or more of the sections being divided into a plurality of subsections.

20. The grid of claim 18, wherein one or more of the sections are divided into a plurality of subsections.
